(19) United States
(12) Patent Application Publication
(10) Pub. No.: US 2023/0392213 A1
(43) Pub. Date: Dec. 7, 2023

(54) COMPOSITIONS AND METHODS FOR TUMOR CHARACTERIZATION

(71) Applicant: The General Hospital Corporation, Boston, MA (US)

(72) Inventors: Gad GETZ, Boston, MA (US); Yosef MARUVKA, Boston, MA (US)

(73) Assignee: The General Hospital Corporation, Boston, MA (US)

(21) Appl. No.: 18/313,091

(22) Filed: May 5, 2023

Related U.S. Application Data
(63) Continuation of application No. PCT/US2021/058241, filed on Nov. 5, 2021.
(60) Provisional application No. 63/111,415, filed on Nov. 9, 2020.
(60) Provisional application No. 63/110,853, filed on Nov. 6, 2020.

(51) Int. Cl. C12Q 1/6886; C07K 16/28

(52) U.S. Cl. CPC C12Q 1/6886; C07K 16/2818; C07K 16/2827; C12Q 2600/118; C12Q 2600/156

(57) ABSTRACT

The invention provides methods for characterizing microsatellite instability in biological samples and for selecting a treatment for a subject having a neoplasia.

### CROSS-REFERENCE TO RELATED APPLICATIONS

This application is a continuation under 35 U.S.C. § 111(a) of PCT International Patent Application No. PCT/US2021/058241, filed Nov. 5, 2021, designating the United States and published in English, which claims priority to and the benefit of U.S. Provisional Application No. 63/111,415, filed Nov. 9, 2020, and U.S. Provisional Application No. 63/110,853, filed Nov. 6, 2020, the entire contents of each of which are incorporated by reference herein.

### SEQUENCE LISTING

The present application contains a Sequence Listing which has been submitted electronically in XML format following conversion from the originally filed TXT format.

The content of the electronic XML Sequence Listing, (Date of creation: May 4, 2023; Size: 7,464 bytes; Name: 167741-028005US-Sequence_Listing.xml), and the original TXT format, is herein incorporated by reference in its entirety.

### BACKGROUND

Microsatellites (“MS(s)”) or microsatellite DNA are genomic regions containing tandem sequence repeats. Generally, microsatellites are tracts of variable-length repeats (generally repeated 5-50 times) of short DNA motifs (ranging in length from 1-6 or more base pairs). Microsatellites may encompass a variety of low complexity sequences, however, most MSs are mono- or di-nucleotide repeats. Microsatellites occur at thousands of locations within an organism's genome, which are distributed throughout the genome. MSs are abundant in nontranscribed regions of the human genome but may also occur in exons and untranslated regions. In the germline, rates of insertions and deletions (indels) in MSs are significantly higher than rates of single-nucleotide substitutions elsewhere in the genome (e.g., about 10⁴ to 10³ compared to about 10⁸ per locus per generation). The increased mutation rate within MS indels is thought to arise because of DNA polymerase slippage during replication, which leads to changes in the number of repeats. MS indels frequently result in frameshift mutations, which can be mutagenic by altering protein expression and/or function.

Tumor microsatellite instability (“MSI”) occurs when one or more MS regions have dramatically higher numbers of MS indels, owing to a loss of normal mismatch repair (MMR) function. Tumors with MS regions that do not display dramatically higher numbers of MS indels are generally referred to as microsatellite stable (“MSS”). Although the MSI phenotype has been observed across many tumor types, it appears to be most common in colon adenocarcinoma (COAD), stomach adenocarcinoma (STAD), and uterine corpus endometrial carcinoma (UCEC). The ability to properly classify tumors as either MSI or MSS has very important prognostic and therapeutic implications. Unfortunately, many clinical centers attempt to identify MSI/MSS tumors based on low throughput PCR- or immunohistochemistry-based testing technologies, and the repetitive nature of MSs makes them challenging to analyze via current sequencing methodologies. In view of the foregoing, there is an urgent unmet need for compositions and methods for classifying and treating MSI tumors.

### SUMMARY OF THE DISCLOSURE

The invention provides methods for characterizing microsatellite instability in biological samples and for selecting a treatment for a subject having a neoplasia. Other features and advantages of the invention will be apparent from the detailed description, and from the claims.

In one aspect, the invention features a method for characterizing microsatellite instability in a biological sample. The method involves comparing the distribution of insertions and deletions in sequencing data obtained from the biological sample over a plurality of microsatellite indels across a genome. The distribution of insertions vs. deletions present in the genome indicates the presence or absence of microsatellite instability in the biological sample.

In another aspect, the invention features a method for treating a selected patient having a neoplasia characterized as having microsatellite instability. The method involves administering to the selected patient an immune checkpoint blockade therapeutic. The patient is selected by comparing the distribution of insertions and deletions in sequencing data obtained from a biological sample of the subject over a plurality of microsatellite indels across a genome. The distribution of insertions vs. deletions present in the genome indicates the presence or absence of microsatellite instability in the biological sample.

In any of the above aspects, or embodiments thereof, the checkpoint blockade therapeutic is a PD-1/PD-L1 inhibitor. In any of the above aspects, or embodiments thereof, the checkpoint blockade theraptutic contains an antibody. In any of the above aspects, or embodiments thereof, the PD-1/PD-L1 inhibitor contains nivolumab, pembrolizumab, atezolizumab, durvalumab, and/or avelumab. In any of the above aspects, or embodiments thereof, the checkpoint blockade therapeutic contains pembrolizumab.

In any of the above aspects, or embodiments thereof, the biological sample contains cell free DNA (cfDNA) and/or tissue. In any of the above aspects, or embodiments thereof, the biological sample contains a neoplasia or tumor sample. In any of the above aspects, or embodiments thereof, the biological sample contains tumor DNA. In embodiments, the cell free DNA contains between about 0.1% and about 3% tumor DNA.

In any of the above aspects, or embodiments thereof, the sequencing is low coverage whole-genome sequencing, low coverage whole-exome sequencing, or targeted Next-Generation Sequencing. In any of the above aspects, or embodiments thereof, the sequence coverage is less than about 1×. In any of the above aspects, or embodiments thereof, the sequencing coverage is between about 0.1× and 0.5×.

In any of the above aspects, or embodiments thereof, the biological sample is derived from a subject having or at risk of having a cancer. In embodiments, the cancer is a colorectal, stomach, or endometrial cancer. In embodiments, the cancer is selected from one or more of a colon adenocarcinoma, a stomach adenocarcinoma, or a uterine corpus endometrial carcinoma. In embodiments, the cancer is a breast, adrenal, or cervical cancer.

In any of the above aspects, or embodiments thereof, the subject has or is at risk for developing Lynch syndrome. In any of the above aspects, or embodiments thereof, the subject has or is at risk for developing minimal residual disease. In any of the above aspects, or embodiments thereof, the subject has or is at risk for developing a relapse of a tumor.

In any of the above aspects, or embodiments thereof, the method does not involve comparing sequence data to a matched normal sample.

In any of the above aspects, or embodiments thereof, the comparing involves calculating a ratio between insertions and deletions. In any of the above aspects, or embodiments thereof, the comparing involves calculating a log likelihood ratio (LLR) of the presence or absence of microsatellite instability in the sample. In embodiments, the log likelihood ratio is an MSI score calculated according to the following formula:

$$MSI score = log ( \frac{1}{\vert {Ω}_{T} \vert} \cdot {\sum }_{{r}_{i} ∈ Ω} \frac{{L}_{q}^{MSI} ( {r}_{i} )}{{L}_{q}^{MSS} ( {r}_{i} )} ) , \qquad ( 2 )$$

where rᵢ is the i'th read, Ω is the set of reads included in the analysis, Ω_{T} this the total set of reads and L is the likelihood that a sample is MSI/MSS given the i'th read.

In any of the above aspects, or embodiments thereof, a log likelihood ratio above a threshold value indicates the presence of microsatellite instability in the biological sample. In embodiments, the threshold value is at least about log(15).

In any of the above aspects, or embodiments thereof, the method involves the simultaneous detection of somatic indels in about 23 million microsatellite loci across an entire genome and/or exome.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “immune chekpoint inhibitor” is meant an agent that inhibits an immune checkpoint protein.

By “PD-1 polypeptide” or “programmed cell death 1 polypeptide” is meant a PD-1 protein or fragment thereof, capable of being activated by PDL-1 and functioning in regulating an immune response and having at least about 85% amino acid sequence identity to GenBank Accession No. AAC51773.1. An exemplary PD-1 amino acid sequence from Homo Sapiens is provided below (GenBank Accession No. AAC51773.1):

(SEQ ID NO: 1)
MQIPQAPWPVVWAVLQLGWRPGWELDSPDRPWNPPTFFPALLVVTEGDN
ATFTCSESNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVESVDYGELDFQWREKTPEPPVPCVPEQ
TEYATIVFPSGMGTSSPARRGSADGPRSAQPLRPEDGHCSWPL.

By “PD-1 polynucleotide” is meant a nucleic acid molecule encoding a PD-1 polypeptide, as well as the introns, exons, and regulatory sequences associated with its expression, or fragments thereof. In embodiments, an PD-1 polynucleotide is the genomic sequence, mRNA, or gene associated with and/or required for PD-1 expression. An exemplary PD-1 nucleotide sequence from Homo Sapiens is provided below (GenBank Accession No. U64863.1):

(SEQ ID NO: 2)
ATGCAGATCCCACAGGCGCCCTGGCCAGTCGTCTGGGCGGTGCTACAAC
TGGGCTGGCGGCCAGGATGGTTCTTAGACTCCCCAGACAGGCCCTGGAA
CCCCCCCACCTTCTTCCCAGCCCTGCTCGTGGTGACCGAAGGGGACAAC
GCCACCTTCACCTGCAGCTTCTCCAACACATCGGAGAGCTTCGTGCTAA
ACTGGTACCGCATGAGCCCCAGCAACCAGACGGACAAGCTGGCCGCCTT
CCCCGAGGACCGCAGCCAGCCCGGCCAGGACTGCCGCTTCCGTGTCACA
CAACTGCCCAACGGGCGTGACTTCCACATGAGCGTGGTCAGGGCCCGGC
GCAATGACAGCGGCACCTACCTCTGTGGGGCCATCTCCCTGGCCCCCAA
GGCGCAGATCAAAGAGAGCCTGCGGGCAGAGCTCAGGGTGACAGAGAGA
AGGGCAGAAGTGCCCACAGCCCACCCCAGCCCCTCACCCAGGCCAGCCG
GCCAGTTCCAAACCCTGGTGGTTGGTGTCGTGGGCGGCCTGCTGGGCAG
CCTGGTGCTGCTAGTCTGGGTCCTGGCCGTCATCTGCTCCCGGGCCGCA
CGAGGGACAATAGGAGCCAGGCGCACCGGCCAGCCCCTGAAGGAGGACC
CCTCAGCCGTGCCTGTGTTCTCTGTGGACTATGGGGAGCTGGATTTCCA
GTGGCGAGAGAAGACCCCGGAGCCCCCCGTGCCCTGTGTCCCTGAGCAG
ACGGAGTATGCCACCATTGTCTTTCCTAGCGGAATGGGCACCTCATCCC
CCGCCCGCAGGGGCTCAGCCGACGGCCCTCGGAGTGCCCAGCCACTGAG
GCCTGAGGATGGACACTGCTCTTGGCCCCTCTGA.

By “PDL-1 polypeptide” or “programmed cell death 1 ligand 1” is meant an a PDL-1 protein or fragment thereof, capable of activating PD-1 and having at least about 85% amino acid sequence identity to Genbank Accession No. AAP13470.1. An exemplary PDL-1 amino acid sequence from Homo Sapiens is provided below (GenBank Accession No. AAP13470.1):

(SEQ ID NO: 3)
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLENVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET.

By “PDL-1 polynucleotide” is meant a nucleic acid molecule encoding a PDL-1 polypeptide, as well as the introns, exons, and regulatory sequences associated with its expression, or fragments thereof. In embodiments, a PDL-1 polynucleotide is the genomic sequence, mRNA, or gene associated with and/or required for PDL-1 expression. An exemplary PDL-1 nucleotide sequence from Homo Sapiens is provided below (GenBank Accession No. AY254342.1):

(SEQ ID NO: 4)
ATGAGGATATTTGCTGTCTTTATATTCATGACCTACTGGCATTTGCTGA
ACGCATTTACTGTCACGGTTCCCAAGGACCTATATGTGGTAGAGTATGG
TAGCAATATGACAATTGAATGCAAATTCCCAGTAGAAAAACAATTAGAC
CTGGCTGCACTAATTGTCTATTGGGAAATGGAGGATAAGAACATTATTC
AATTTGTGCATGGAGAGGAAGACCTGAAGGTTCAGCATAGTAGCTACAG
ACAGAGGGCCCGGCTGTTGAAGGACCAGCTCTCCCTGGGAAATGCTGCA
CTTCAGATCACAGATGTGAAATTGCAGGATGCAGGGGTGTACCGCTGCA
TGATCAGCTATGGTGGTGCCGACTACAAGCGAATTACTGTGAAAGTCAA
TGCCCCATACAACAAAATCAACCAAAGAATTTTGGTTGTGGATCCAGTC
ACCTCTGAACATGAACTGACATGTCAGGCTGAGGGCTACCCCAAGGCCG
AAGTCATCTGGACAAGCAGTGACCATCAAGTCCTGAGTGGTAAGACCAC
CACCACCAATTCCAAGAGAGAGGAGAAGCTTTTCAATGTGACCAGCACA
CTGAGAATCAACACAACAACTAATGAGATTTTCTACTGCACTTTTAGGA
GATTAGATCCTGAGGAAAACCATACAGCTGAATTGGTCATCCCAGAACT
ACCTCTGGCACATCCTCCAAATGAAAGGACTCACTTGGTAATTCTGGGA
GCCATCTTATTATGCCTTGGTGTAGCACTGACATTCATCTTCCGTTTAA
GAAAAGGGAGAATGATGGATGTGAAAAAATGTGGCATCCAAGATACAAA
CTCAAAGAAGCAAAGTGATACACATTTGGAGGAGACGTAA.

By “nivolumab” is meant an antibody that binds PD-1 and/or PD-L1. In embodiments, the antibody is a humanized antibody for use in cancer immunotherapy. In embodiments, the cancer immunotherapy is used to treat a cancer or neoplasm.

By “pembrolizumab” is meant an antibody that binds PD-1 and/or PD-L1. In embodiments, the antibody is a humanized antibody for use in cancer immunotherapy. In embodiments, the cancer immunotherapy is used to treat a cancer or neoplasm.

By “atezolizumab” is meant an antibody that binds PD-1 and/or PD-L1. In embodiments, the antibody is a humanized antibody for use in cancer immunotherapy. In embodiments, the cancer immunotherapy is used to treat a cancer or neoplasm.

By “durvalumab” is meant an antibody that binds PD-1 and/or PD-L1. In embodiments, the antibody is a humanized antibody for use in cancer immunotherapy. In embodiments, the cancer immunotherapy is used to treat a cancer or neoplasm.

By “avelumab” is meant an antibody that binds PD-1 and/or PD-L1. In embodiments, the antibody is a humanized antibody for use in cancer immunotherapy. In embodiments, the cancer immunotherapy is used to treat a cancer or neoplasm.

Unless specifically stated or obvious from context, as used herein, the term “about” is understood as within a range of normal tolerance in the art. Unless otherwise clear from context, all numerical values provided herein are modified by the term “about.”

The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally, or alternatively, in some embodiments, administration is parenteral. In some embodiments, administration is intravenous.

By “agent” is meant any small compound (e.g., small molecule), antibody, nucleic acid molecule, or polypeptide, or fragments thereof. In one embodiment, an agent of the disclosure is a PD1/PD-L1 immune checkpoint blockade therapy (e.g., immuno-oncology therapeutic).

As used herein, the term “algorithm” refers to any formula, model, mathematical equation, algorithmic, analytical or programmed process, or statistical technique or classification analysis that takes one or more inputs or parameters, whether continuous or categorical, and calculates an output value, index, index value or score. Examples of algorithms include but are not limited to ratios, sums, regression operators such as exponents or coefficients, biomarker value transformations and normalizations (including, without limitation, normalization schemes that are based on clinical parameters such as age, gender, ethnicity, etc.), rules and guidelines, statistical classification models, statistical weights, and neural networks trained on populations or datasets. Also, of use in the context of MSI as described herein are linear and non-linear equations and statistical classification analyses to determine the relationship between the presence of indels detected at specific MS loci in the genome of a subject's tumor sample.

The term “cancer” refers to a malignant neoplasm (Stedman's Medical Dictionary, ed.; Hensyl ed.; Williams & Wilkins: Philadelphia, 1990). Exemplary cancers include, but are not limited to, colon adenocarcinoma (COAD), esophageal carcinoma (ESCA), rectal adenocarcinoma (READ), stomach adenocarcinoma (STAD) and uterine corpus endometrial carcinoma (UCEC). It is also contemplated within the scope of the disclosure that the techniques herein may be applied to detect MSI in liquid tumors such as, for example, leukemia and lymphoma.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result. In embodiments, a reference is a subject or a sample from a subject that does not have constitutional mismatch repair deficiency (CMMRD), a subject who is mismatch repair (MMR) proficient, and/or a subject who is polymerase proofreading proficient. In embodiments, the reference is a matched normal sample, where in some instances the matched normal sample is a sample from a healthy subject and/or a subject that does not have constitutional mismatch repair deficiency (CMMRD), a subject who is mismatch repair (MMR) proficient, and/or a subject who is polymerase proofreading proficient.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

By “homopolymers(s)” is meant a microsatellite (MS) that is a mononucleotide repeat of at least 6 bases (e.g., a stretch of at least 6 consecutive A, C, T or G residues in the DNA). A “homopolymer region” is a MS region in which the microsatellite is a homopolymer. A “homopolymer subregion” refers to a homopolymer microsatellite located within a larger genomic region (e.g., a homopolymer region).

As used herein, the term “indel” refers to a mutation in a nucleic acid in which one or more nucleotides are either inserted or deleted, resulting in a net gain or loss of nucleotides that can include any combination of insertions and deletions. Aberrant homopolymer lengths often result from indels.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

As used herein, “microsatellite (MS)” refers to a genetic locus comprising a short (e.g., 1-20, 1-15, 1-10, 1-9, 1-8, 1-7, 1-6, 1-5, 1-4, 1-3, 1-2, etc.), tandemly repeated sequence motifs comprising a minimal total length of about 6 bases. A “mononucleotide microsatellite” or refers to a genetic locus comprising a repeated single nucleotide (e.g., poly-A) and is a specific subclass of MSs. A “dinucleotide microsatellite” refers to a genetic locus comprising a motif of two nucleotides that are tandemly repeated, a “trinucleotide microsatellite” refers to a genetic locus comprising three nucleotides that are tandemly repeated, and a “tetranucleotide microsatellite” refers to a genetic locus comprising a motif of four nucleotides that are tandemly repeated. Additional microsatellite motifs can comprise pentanucleotide and hexanucleotide repeats. A “monomorphic microsatellite” is one in which all (or substantially all) individuals, particularly all individuals of a given population, share the same number of repeat units, which is in contrast to a “polymorphic microsatellite,” which is used to refer to microsatellites in which more than about 1% of individuals in a given population display a different number of repeat units in at least of their alleles. When analyzing MS, one may look at genomic DNA of a sample (e.g., genomic DNA of a tumor cell). “Microsatellite region” refers to the genomic context in which a particular microsatellite resides (i.e., the particular genomic region containing the MS).

As used herein, “microsatellite instability (MSI)” refers to a clonal or somatic change in the number of repeated DNA nucleotide units in MSs such as, for example, insertions and deletions (indels). The term “microsatellite stable (MSS)” refers to MSs that do not display a clonal or somatic change in the number of repeated DNA nucleotide units in the respective MSs. In some embodiments detecting MSI in a tumor or cancer cell sample may include classifying MSI or MSS status in the tumor or cancer cell, in which case the method may include a classification step as described herein.

By “neoplasia” is meant a disease or disorder characterized by excess proliferation or reduced apoptosis. Illustrative neoplasms for which the disclosure can be used include, but are not limited to, breast cancer, esophageal cancer, pancreatic cancer, colorectal cancer, hepatocellular cancer, bladder cancer, luminal and non-luminal bladder cancer, basal bladder cancer, muscle-invasive bladder cancer, and non-muscle-invasive bladder cancer, pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). In embodiments, the neoplasia may be colon adenocarcinoma (COAD), stomach adenocarcinoma (STAD), and uterine corpus endometrial carcinoma (UCEC). In embodiments, the neoplasia may be a liquid tumor such as, for example, leukemia or lymphoma.

As used herein, the term “next-generation sequencing (NGS)” refers to a variety of high-throughput sequencing technologies that parallelize the sequencing process, producing thousands or millions of sequence reads at once. NGS parallelization of sequencing reactions can generate hundreds of megabases to gigabases of nucleotide sequence reads in a single instrument run. Unlike conventional sequencing techniques, such as Sanger sequencing, which typically report the average genotype of an aggregate collection of molecules, NGS technologies typically digitally tabulate the sequence of numerous individual DNA fragments (sequence reads discussed in detail below), such that low frequency variants (e.g., variants present at less than about 10%, 5% or 1% frequency in a heterogeneous population of nucleic acid molecules) can be detected. The term “massively parallel” can also be used to refer to the simultaneous generation of sequence information from many different template molecules by NGS. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (ion Torrent); and DNA nanoball sequencing (Complete Genomics). Descriptions of certain NGS platforms can be found in the following: Shendure, et al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3):333-43; and Zhang et al., “The impact of next-generation sequencing on genomics,” J Genet Genomics, 201, 38(3): 95-109.

As used herein, the term “subject” includes humans and mammals (e.g., mice, rats, pigs, cats, dogs, horses, and the like). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease or condition in a mammal, particularly in a human, and includes: (a) preventing the disease from occurring in a subject which can be predisposed to the disease but has not yet been diagnosed as having it; (b) inhibiting the disease, e.g., arresting its development; and (c) relieving the disease, e.g., causing regression of the disease.

The phrase “pharmaceutically acceptable carrier” is art recognized and includes a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977):1-19, incorporated herein by reference. The salts can be prepared in situ during the final isolation and purification of the compounds (e.g., FDA-approved compounds) of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound (e.g., an FDA-approved compound where administered to a human subject) or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the certain compounds of the present application which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the application. The term “prodrug” refers to compounds that are rapidly transformed in vivo to yield the parent compound of an agent of the instant disclosure, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, (1987), both of which are incorporated herein by reference.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “pharmaceutically acceptable salts, esters, amides, and prodrugs” as used herein refers to those carboxylate salts, amino acid addition salts, esters, amides, and prodrugs of the compounds of the present disclosure which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the disclosure.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “salts” refers to the relatively non-toxic, inorganic and organic acid addition salts of compounds of the present disclosure. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, tetramethylammonium, tetramethylammonium, methlyamine, dimethlyamine, trimethlyamine, triethlyamine, ethylamine, and the like. (See, for example, S. M. Barge et al., “Pharmaceutical Salts,” J. Pharm. Sci., 1977, 66:1-19 which is incorporated herein by reference.).

A “therapeutically effective amount” of an agent described herein is an amount sufficient to provide a therapeutic benefit in the treatment of a condition or to delay or minimize one or more symptoms associated with the condition. A therapeutically effective amount of an agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In addition, the materials, methods, and examples are illustrative only and not intended to be limiting.

These and other embodiments are disclosed or are obvious from and encompassed by, the following Detailed Description. Although methods and materials similar or equivalent to those described herein can be used in the practice or testing of the present disclosure, suitable methods and materials are described below.

### BRIEF DESCRIPTION OF THE DRAWINGS

FIGS. 1A-1E: MS-indel distribution from WGS. A MS-indels distribution of a typical MSS case. MS-indels found in /WGS of MSI case. B. MS-indels distribution of a typical MSI case. MS-indels in WGS of MSS case. C-D Similar to A-B but instead of an inferred indel distribution reads length distribution is presented. C. Reads with indels in WGS of MSI case. D. Reads with indels in WGS of MSI case. E. MS-indels signatures for a typical MSS sample (left panel) and a typical MSI sample (right panel)

FIGS. 2A-2E. Log-likelihood ratio. A. Reads length of distribution. B. A schematic description of the MSI classification process. C. MSIness values for different fractions of mixing the DNA of MSI cell-lines with normal DNA. D. MSIness score for prepared mixtures and cfDNA of colon cases using 0.5×WGS sequencing, 7 with known current MSI tumors, 22 with known current MSS tumors as a training set, the rest Lynch cases with and without active tumor, and sporadic MSI cases were used as validation. In FIG. 2D, the following calculations were used: A) Log P(MSI)=sum_i log (P(MSI|read_i)) (assume reads are independent)=sum_i[log P(read_i|MSI)*P(MSI)/P(read_i)]=sum_i[log P(read_i|MSI)*P(MSI)]+K; and B) Log P(MSI)/P(MSS)=sum_i[log P(read_i|MSI)−log P(read_i|MSI)]+log P(MSI)−log P(MSS). The calculation of a per sample MSI score can be completed using the following formula:

$$S = {\sum }_{{r}_{i}} {w}_{Locus} log ( \frac{{P}_{Locus}^{MSI} ( {r}_{i} )}{{P}_{Locus}^{MSS} ( {r}_{i} )} ) .$$

E. is a plot presenting results from MSI detection in low cancer fraction from ultra low pass whole genome sequencing (0.5×coverage).

FIG. 3. Tumor classification. MSIDetect score using DFCI OncoPanel sequencing data with clinical annotation of MSI status. The MSI-score threshold was determined for each version of the OncoPanel assay separately.

FIG. 4 provides a plot and a bar graph providing a comparison of MSIDetect with other MSI NGS classifiers. A data set of 18 MSI and 17 MSS UCED WES samples was analyzed using the indicated classifiers (MSIDetect; MSI SEQ; MSISensor; and MANTIS). The left panel of FIG. 4 presents the MSIness of the MSI and MSS samples. The test set was uterine corpus endometrial cancer (UCEC).

FIG. 5. Number of MS-indels in MSI vs. MSS. In the left panel all the samples are combined into one statistical analysis. They are divided into each tumor type in the other panels, in all tumor types the difference is significant (P-value, Wilcoxon Rank test).

FIG. 6. Separation of MSI and MSS samples based on reads ratio (RR score=log₁₀(ratio of 5 bp deletions/1 bp insertions). Same as FIG. 1E but each tumor type was normalized to the mean of the normal samples (both from MSI and MSS) and rescaled by the standard deviation of the normal samples.

FIG. 7. Separation heat map for stomach cancer scanning. The value in each locus-length and deletion size pair is the difference between the mean normalized score of the MSI cases and the MSS cases.

FIG. 8. Reads ratio score—the mean of the normalized reads ratio score over the most variable events.

FIG. 9. Difference of the reads ratio (RR) score (RR score=log₁₀(ratio of 5 bp deletions/1 bp insertions) between a tumor and its associated normal cells.

FIG. 10. A comparison of the Log likelihood ratio distribution of an MSI sample (TCGA-CG-4442) and an MSS sample (TCGA-CG-4443). The score corresponding to the grey dashed line was used as a threshold. In embodiments, the threshold is used to calculate the LLR score. In embodiments, the threshold is used to separate MSS samples from MSI samples. In embodiments, the threshold is log(15), where the base of the logarhythm is 10.

FIG. 11. Log-likelihood ratio score for the TCGA WGS samples.

FIG. 12. Combining the performance of the two score methods used, the reads ratio score and the Log-likelihood ratio score. The grey dashed lines are the highest value of the normal samples (NA12878).

FIG. 13 MS-indels distributions. Top panel of FIG. 13: MS-indels distribution for TCGA-A6-A565. Same as FIGS. 1A-B. Bottom panel of FIG. 13: Same as FIGS. 1A-B for the sample TCGA-BS-A0TE.

The following detailed description, given by way of example, but not intended to limit the disclosure solely to the specific embodiments described, may best be understood in conjunction with the accompanying drawings.

### DETAILED DESCRIPTION OF THE DISCLOSURE

The invention features compositions and methods that are useful for characterizing microsatellite instability in biological samples and for selecting a treatment for a subject having a neoplasia.

The invention is based at least in part upon the discovery described in the examples provided herein below that microsatellite instability or mismatch repair deficient cancers can be detected using cell-free DNA from a blood sample. The Examples provided herein demonstrate the efficacy of a technique, termed “MSIDetect,” which provides a sensitive tool for detecting tumors with microsatellite instability (MSI) from cell-free DNA (cfDNA) or tissue samples, using low coverage whole-genome sequencing (˜0.5×) or targeted panels. MSIDetect does not require a germline control and can detect MSI with as low as 0.1% of cancer DNA in the cfDNA. MSIDetect was applied to 50 cfDNA and ˜1,308 tumor samples, with and without MSI, and demonstrated its high sensitivity and specificity both when using cfDNA and panel data.

### Microsatellites

Microsatellites (MSs), also known as short tandem repeats, are regions of the genome characterized by repetition of a short sequence motif (usually 1-6 bp), e.g. AAAAAA or ACACACACAC (SEQ ID NO: 5). Not intending to be bound by theory, MSs are abundant in non-transcribed regions of the human genome, but also occur in exons and untranslated regions (UTRs) with a similar frequency. In the germline, rates of insertions and deletions (indels) in MSs are significantly higher than rates of single nucleotide substitutions elsewhere in the genome (10⁻⁴-10⁻³ compared to ˜10⁻⁸ per locus per generation, respectively). The increased indel mutation rate within MSs is thought to arise due to DNA polymerase slippage during replication of repetitive sequences, leading to changes in the number of repeats. MS indels frequently result in frameshift mutations and can therefore dramatically alter protein function by changing the amino acid sequence and/or introducing premature stop codons.

Given their prevalence and relatively high mutation rate, it is perhaps not surprising that microsatellites have been widely implicated in human disease. More than 40 hereditary diseases are caused by germline MS indels, including Huntington's disease and fragile X syndrome. In addition, many cancer genes contain MS loci, and in some cases, somatic MS indels have been causally implicated in cancer. Tumors with microsatellite instability (MSI) have dramatically increased numbers of MS indels owing to loss of normal mismatch repair (MMR) function. Although the MSI phenotype has been observed across tumor types, it appears to be most common in colon adenocarcinoma (COAD), stomach adenocarcinoma (STAD), and uterine corpus endometrial carcinoma (UCEC). Given the important prognostic and therapeutic implications of MSI status, many clinical centers perform routine PCR- or immunohistochemistry-based MSI testing for these tumor types.

Despite their potential biological significance, somatic MS indels have not been systematically analyzed in cancer due to challenges associated with their detection via current next-generation sequencing (NGS) technologies. Only NGS reads that span the entire length of a MS and include sufficient 5′ and 3′ flanking sequences can be used to infer the number of repeated motifs in the MS. In addition, the PCR amplification step that is performed during NGS can itself suffer from DNA polymerase slippage events similar to those that lead to MS indels in vivo, thereby creating NGS artifacts that may be falsely interpreted as MS indels. The frequency of such sequencing errors varies across MS loci and depends on parameters such as the specific MS motif and the number of repeats. Therefore, novel methods utilizing principled statistical modeling and noise estimation are required to accurately identify true MS indel events.

### Microsatellite Stability Classification

Applying MSMutSig across 6,747 tumors from 20 different tumor types identified 7 genes with significant MS indel hotspots: ACVR2A, RNF43, DOCK3, MSH3, ESRP 1, PRDM2 and/or JAK1. In the four genes that have been previously implicated in cancer (ACVR2A, RNF43, JAK1 and MSH3), previously unreported MS indels events were identified. Three of the genes with significant loci—DOCK3, PRDM2 and ESRP 1—had not been previously listed as cancer genes. MS indels in DOCK3, a negative regulator of the WNT pathway, were mutually exclusive with mutations in CTNNB1. MS indels in ESRP 1, an RNA processing gene, correlated with alternative splicing of FGFR2, an event associated with the epithelial-to-mesenchymal transition.

The present disclosure relates to detecting microsatellite indels in a cancer patient and/or an individual at risk for cancer. In one embodiment, the disclosure relates to detecting one or more indels in one or more genes with significant microsatellite stable indel hotspots. In an advantageous embodiment, the one or more genes with significant microsatellite stable indel hotspots include, but are not limited to, ACVR2A, RNF43, DOCK3, MSH3, ESRP 1, PRDM2 and/or JAK1 genes.

The present disclosure relates to achieving classification by using whole genome or whole exome data. The classification relies both on the fact that high MS instability (MSI-H) contains a large fraction of the MSI loci mutated, as well as the fact that the type of MS indels in MSI-H cases differ from those in microsatellite stable (MSS) cases. For example, MSI tumors tend to have more one-base deletion in medium size loci (8-15 bases), while non-MSI cases, even if they contain many MS indels, have a more uniform ratio of deletions and insertions, and they do not have this bias to medium sized loci.

The present disclosure relates to a method of identifying and selecting a subject with a cancer or tumor with high microsatellite instability (MSI-H) (as opposed to low microsatellite instability (MSI-L) or a microsatellite stable (MSS) cancer or tumor) which may comprise detecting a limited plurality of not more than 40, 30, 20 or 10 microsatellite indels associated with the MSI-H cancer or tumor (but not a MSI-L cancer or tumor), in a nucleic acid sample from the subject's cancer or tumor, wherein the limited plurality of not more than 40 or 30 or or 10 microsatellite indels that are highly mutated in MSI (MSI-H) cancers, but have a low indel rate in an MSI-L or MSS cancer or tumor, and/or may be identified by a limited plurality set of indels are selected by MSMuTect, and wherein the subject has an MSI-H cancer or tumor if all or at least 39, 35, 30 or 20 of the 40 of the limited plurality of MS indels is present in the nucleic acid sample from the subject's cancer or tumor.

The methods typically conducted on a biological sample selected from for example tumor biopsy samples, blood samples (isolation and enrichment of shed tumor cells), stool biopsies, sputum, chromosome, pleural fluid, peritoneal fluid, buccal spears or biopsy or urine.

### Detecting Microsatellite Indels

The present disclosure also involves other methods for detecting the MS indels of the present disclosure. Whole genome or whole exome sequencing is preferred; however, other methods of sequencing and hybridization are also envisioned. In embodiments, the sequencing is to a coverage of about or at least about 0.001, 0.01, 0.05, 0.1, 0.2, 0.3, 0.4, 0.5, 0.75, 1, 2, 3, 4, 5, 6, 7, 8, 9, or 10×, where a sequencing coverage of 0.01 indicates that a DNA sample has been sequenced such that the amount of DNA sequenced is equivalent in size to about 1% of the corresponding genome from which the DNA sample is derived. In embodiments, the sequencing is to a coverage of no more than about 0.001, 0.01, 0.05, 0.1, 0.2, 0.3, 0.4, 0.5, 0.75, 1, 2, 3, 4, 5, 6, 7, 8, 9, or 10×.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this disclosure it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

For purpose of this disclosure, the term “amplification” means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway. Amplification may involve thermocycling or isothermal amplification (such as through the methods RPA or LAMP). Cross-linking may involve overlap-extension PCR or use of ligase to associate multiple amplification products with each other.

RNA sequencing (RNA-Seq) is a powerful tool for transcriptome profiling but is hampered by sequence-dependent bias and inaccuracy at low copy numbers intrinsic to exponential PCR amplification. To mitigate these complications to allow truly digital RNA-Seq, a large set of barcode sequences is added in excess, and nearly every cDNA molecule is uniquely labeled by random attachment of barcode sequences to both ends (Shiroguchi K, et al. Proc Natl Acad Sci USA. 2012 Jan. 24; 109(4):1347-52). After PCR, paired-end deep sequencing is applied to read the two barcodes and cDNA sequences. Rather than counting the number of reads, RNA abundance is measured based on the number of unique barcode sequences observed for a given cDNA sequence (Shiroguchi K, et al. Proc Natl Acad Sci USA. 2012 Jan. 24; 109(4):1347-52). The barcodes may be optimized to be unambiguously identifiable, even in the presence of multiple sequencing errors. This method allows counting with single-copy resolution despite sequence-dependent bias and PCR-amplification noise and is analogous to digital PCR but amendable to quantifying a whole transcriptome (Shiroguchi K, et al. Proc Natl Acad Sci U.S.A. 2012 Jan. 24; 109(4):1347-52).

Fixation of cells or tissue may involve the use of cross-linking agents, such as formaldehyde, and may involve embedding cells or tissue in a paraffin wax or polyacrylamide support matrix (Chung K, et al. Nature. 2013 May 16; 497(7449): 322-7).

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are well established in the art and are taught in U.S. Pat. No. 5,210,015.

Sequencing may be performed on any high-throughput platform with read-length (either single- or paired-end) sufficient to cover both template and cross-linking event UID's. Methods of sequencing oligonucleotides and nucleic acids are well known in the art (see, e.g., WO93/23564, WO98/28440 and WO98/13523; U.S. Pat. Nos. 5,525,464; 5,202,231; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference).

The present disclosure may be applied to (1) single-cell transcriptomics: cDNA synthesized from mRNA is barcoded and cross-linked during in situ amplification, (2) single-cell proteomics: cDNA or DNA synthesized from RNA- or DNA-tagged antibodies of one or multiple specificities maps the abundance and distributions of different protein-antigens and (3) whole-tissue transcriptomic/proteomic mapping (molecular microscopy or VIPUR microscopy): using the frequency of cross-contamination between cells to determine their physical proximity, and via applications (1) single-cell transcriptomics and (2) single-cell proteomics, determining the global spatial distribution of mRNA, protein, or other biomolecules in a biological sample. This may be used, for example, to screen for anti-cancer immunoglobulins (by analyzing co-localization of B-cells and T-cells within affected tissue) for immunotherapy.

As described in aspects of the disclosure, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

Sequence homologies may be generated by any of a number of computer programs known in the art, for example BLAST or FASTA, etc. A suitable computer program for carrying out such an alignment is the GCG Wisconsin Bestfit package (University of Wisconsin, U.S.A; Devereux et al., 1984, Nucleic Acids Research 12:387). Examples of other software than may perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program. % homology may be calculated over contiguous sequences, e.g., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues. Although this is a very simple and consistent method, it fails to take into consideration that, for example, in an otherwise identical pair of sequences, one insertion or deletion may cause the following amino acid residues to be put out of alignment, thus potentially resulting in a large reduction in % homology when a global alignment is performed. Consequently, most sequence comparison methods are designed to produce optimal alignments that take into consideration possible insertions and deletions without unduly penalizing the overall homology or identity score. This is achieved by inserting “gaps” in the sequence alignment to try to maximize local homology or identity. However, these more complex methods assign “gap penalties” to each gap that occurs in the alignment so that, for the same number of identical amino acids, a sequence alignment with as few gaps as possible—reflecting higher relatedness between the two compared sequences—may achieve a higher score than one with many gaps. “Affinity gap costs” are typically used that charge a relatively high cost for the existence of a gap and a smaller penalty for each subsequent residue in the gap. This is the most commonly used gap scoring system. High gap penalties may, of course, produce optimized alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension. Calculation of maximum % homology therefore first requires the production of an optimal alignment, taking into consideration gap penalties. A suitable computer program for carrying out such an alignment is the GCG Wisconsin Bestfit package (Devereux et al., 1984 Nuc. Acids Research 12 p387). Examples of other software than may perform sequence comparisons include, but are not limited to, the BLAST package (see Ausubel et al., 1999 Short Protocols in Molecular Biology, 4th Ed.—Chapter 18), FASTA (Altschul et al., 1990 J. Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60). However, for some applications, it is preferred to use the GCG Bestfit program. A new tool, called BLAST 2 Sequences is also available for comparing protein and nucleotide sequences (see FEMS Microbiol Lett. 1999 174(2): 247-50; FEMS Microbiol Lett. 1999 177(1): 187-8 and the website of the National Center for Biotechnology information at the website of the National Institutes for Health). Although the final % homology may be measured in terms of identity, the alignment process itself is typically not based on an all-or-nothing pair comparison. Instead, a scaled similarity score matrix is generally used that assigns scores to each pair-wise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table, if supplied (see user manual for further details). For some applications, it is preferred to use the public default values for the GCG package, or in the case of other software, the default matrix, such as BLOSUM62.

Alternatively, percentage homologies may be calculated using the multiple alignment feature in DNASIS™ (Hitachi Software), based on an algorithm, analogous to CLUSTAL (Higgins D G & Sharp P M (1988), Gene 73(1), 237-244). Once the software has produced an optimal alignment, it is possible to calculate % homology, preferably % sequence identity. The software typically does this as part of the sequence comparison and generates a numerical result.

Embodiments of the disclosure include sequences (both polynucleotide or polypeptide) which may comprise homologous substitution (substitution and replacement are both used herein to mean the interchange of an existing amino acid residue or nucleotide, with an alternative residue or nucleotide) that may occur e.g., like-for-like substitution in the case of amino acids such as basic for basic, acidic for acidic, polar for polar, etc. Non-homologous substitution may also occur e.g., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as 0), pyriylalanine, thienylalanine, naphthylalanine and phenylglycine.

Hybridization can be performed under conditions of various stringency. Suitable hybridization conditions for the practice of the present disclosure are such that the recognition interaction between the probe and sequences associated with a signaling biochemical pathway is both sufficiently specific and sufficiently stable. Conditions that increase the stringency of a hybridization reaction are widely known and published in the art. See, for example, (Sambrook, et al., (1989); Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A preferred hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present disclosure include any composition detectable by photochemical, biochemical, spectroscopic, immunochemical, electrical, optical or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, ß-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphoimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

Examples of the labeling substance which may be employed include labeling substances known to those skilled in the art, such as fluorescent dyes, enzymes, coenzymes, chemiluminescent substances, and radioactive substances. Specific examples include radioisotopes (e.g., 32P, 14C, 125I, 3H, and 131I), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added.

Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine

The fluorescent label may be a fluorescent protein, such as blue fluorescent protein, cyan fluorescent protein, green fluorescent protein, red fluorescent protein, yellow fluorescent protein or any photoconvertible protein. Colormetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code.

In an advantageous embodiment, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation.

In an advantageous embodiment, agents may be uniquely labeled in a dynamic manner (see, e.g., international patent application serial no. PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

The oligonucleotide tags may be detectable by virtue of their nucleotide sequence, or by virtue of a non-nucleic acid detectable moiety that is attached to the oligonucleotide such as but not limited to a fluorophore, or by virtue of a combination of their nucleotide sequence and the nonnucleic acid detectable moiety.

In some embodiments, a detectable oligonucleotide tag may comprise one or more nonoligonucleotide detectable moieties. Examples of detectable moieties may include, but are not limited to, fluorophores, microparticles including quantum dots (Empodocles, et al., Nature 399:126-130, 1999), gold nanoparticles (Reichert et al., Anal. Chem. 72:6025-6029, 2000), biotin, DNP (dinitrophenyl), fucose, digoxigenin, haptens, and other detectable moieties known to those skilled in the art. In some embodiments, the detectable moieties may be quantum dots. Methods for detecting such moieties are described herein and/or are known in the art.

Thus, detectable oligonucleotide tags may be, but are not limited to, oligonucleotides which may comprise unique nucleotide sequences, oligonucleotides which may comprise detectable moieties, and oligonucleotides which may comprise both unique nucleotide sequences and detectable moieties.

A unique label may be produced by sequentially attaching two or more detectable oligonucleotide tags to each other. The detectable tags may be present or provided in a plurality of detectable tags. The same or a different plurality of tags may be used as the source of each detectable tag may be part of a unique label. In other words, a plurality of tags may be subdivided into subsets and single subsets may be used as the source for each tag.

In some embodiments, a detectable oligonucleotide tag may comprise one or more non-oligonucleotide detectable moieties. Examples of detectable moieties include, but are not limited to, fluorophores, microparticles including quantum dots (Empodocles, et al., Nature 399:126-130, 1999), gold nanoparticles (Reichert et al., Anal. Chem. 72:6025-6029, 2000), biotin, DNP (dinitrophenyl), fucose, digoxigenin, haptens, and other detectable moieties known to those skilled in the art. In some embodiments, the detectable moieties are quantum dots. Methods for detecting such moieties are described herein and/or are known in the art.

Thus, detectable oligonucleotide tags may be, but are not limited to, oligonucleotides which may comprise unique nucleotide sequences, oligonucleotides which may comprise detectable moieties, and oligonucleotides which may comprise both unique nucleotide sequences and detectable moieties.

A unique nucleotide sequence may be a nucleotide sequence that is different (and thus distinguishable) from the sequence of each detectable oligonucleotide tag in a plurality of detectable oligonucleotide tags. A unique nucleotide sequence may also be a nucleotide sequence that is different (and thus distinguishable) from the sequence of each detectable oligonucleotide tag in a first plurality of detectable oligonucleotide tags but identical to the sequence of at least one detectable oligonucleotide tag in a second plurality of detectable oligonucleotide tags. A unique sequence may differ from other sequences by multiple bases (or base pairs). The multiple bases may be contiguous or non-contiguous. Methods for obtaining nucleotide sequences (e.g., sequencing methods) are described herein and/or are known in the art.

In some embodiments, detectable oligonucleotide tags comprise one or more of a ligation sequence, a priming sequence, a capture sequence, and a unique sequence (optionally referred to herein as an index sequence). A ligation sequence is a sequence complementary to a second nucleotide sequence which allows for ligation of the detectable oligonucleotide tag to another entity which may comprise the second nucleotide sequence, e.g., another detectable oligonucleotide tag or an oligonucleotide adapter. A priming sequence is a sequence complementary to a primer, e.g., an oligonucleotide primer used for an amplification reaction such as but not limited to PCR. A capture sequence is a sequence capable of being bound by a capture entity. A capture entity may be an oligonucleotide which may comprise a nucleotide sequence complementary to a capture sequence, e.g. a second detectable oligonucleotide tag. A capture entity may also be any other entity capable of binding to the capture sequence, e.g. an antibody, hapten or peptide. An index sequence is a sequence which may comprise a unique nucleotide sequence and/or a detectable moiety as described above.

### Computer Systems

The present disclosure also relates to a computer system involved in carrying out the methods of the disclosure relating to both computations and sequencing.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

A computer can transform data into various formats for display. A graphical presentation of the results of a calculation (e.g., MSIness score) can be displayed on a monitor, display, or other visualizable medium (e.g., a printout). In some embodiments, data or the results of a calculation may be presented in an auditory form.

### Multiplex Assays

The present disclosure also contemplates multiplex assays. The present disclosure is especially well suited for multiplex assays. For example, the disclosure encompasses use of a SureSelect^{XT,} SureSelect^{XT2} and SureSelect^{QXT} Target Enrichment System for Illumina Multiplexed Sequencing developed by Agilent Technologies (see for example the World Wide Web at (www)agilent.com/genomics/protocolvideos), a SeqCap EZ kit developed by Roche NimbleGen, a TruSeq® Enrichment Kit developed by Illumina and other hybridization-based target enrichment methods and kits that add sample-specific sequence tags either before or after the enrichment step. as well as Illumina HiSeq, MiSeq and NexSeq, Life Technology Ion Torrent. Pacific Biosciences PacBio RSII, Oxford Nanopore MinIon, Promethlon and GridIon and other massively parallel Multiplexed Sequencing PlatformsError! Hyperlink reference not valid.

Usable methods for hybrid selection are described in Melnikov, et al., Genome Biology 12:R73, 2011; Geniez, et al., Symbiosis 58:201-207, 2012; and Matranga, et al., Genome Biology 15:519, 2014). Bait design and hybrid selection was done similarly to a previously published method (see, e.g., Gnirke, et al., Nature biotechnology 27:182-189, 2009, US Patent Publications No. US 2010/0029498, US 2013/0230857, US 2014/0200163, US 2014/0228223, and US 2015/0126377 and International Patent Publication No. WO 2009/099602). Briefly, baits may be designed by first concatenating all consensus sequences (such as LASV) into two single bait sets (such as one for Nigerian clades and another for the Sierra Leone Glade). Duplicate probes, defined as a DNA sequence with 0 mismatches, were removed. The baits sequences were tiled across the genome (such as LASV) creating a probe every 50 bases. Two sets of adapters were used for each bait set. Adapters alternated with each 50 base probe to improve the efficiency of PCR amplification of probes. The oligo array was synthesized on a CustomArray B3 Synthesizer, as recommended by the manufacturer. The oligonucleotides were cleaved-off the array and amplified by PCR with primers containing T7 RNA polymerase promoters. Biotinylated baits were then prepared through in vitro transcription (MEGAshortscript, Ambion). RNA baits for each Glade were prepared separately and mixed at the equal RNA concentration prior to hybridization. Libraries of the genome (such as LASV) were added to the baits and hybridized over a 72 hrs. After capture and washing, libraries were amplified by PCR using the Illumina adapter sequences. Libraries were then pooled and sequenced on the MiSeq platform.

### Cancer Treatments

Methods of inhibiting and/or treating cancer and tumors in individuals with cancer or a predisposition for developing cancer as identified by methods of the disclosure are also contemplated.

The subject has been diagnosed with cancer or is at risk of developing cancer. The subject is a human, dog, cat, horse, or any animal in which a tumor specific immune response is desired. The tumor is any solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas. In an advantageous embodiment, the cancer is an adrenal, breast, cervical, colon, endometrial, rectal or stomach cancer.

The therapeutic agent is for example, a chemotherapeutic agent, radiation, or immunotherapy. Any suitable therapeutic treatment for a particular cancer may be administered. Examples of chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, tamoxifen, taxol, topotecan hydrochloride, trastuzumab, vinblastine, vincristine and vinorelbine tartrate.

For therapeutic use, administration should begin at the detection or surgical removal of tumors. This is followed by boosting doses until at least symptoms are substantially abated and for a period thereafter.

The pharmaceutical compositions for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor. The disclosure provides compositions for parenteral administration which comprise a solution of the peptides and vaccine compositions are dissolved or suspended in an acceptable carrier, preferably an aqueous carrier. A variety of aqueous carriers may be used, e.g., water, buffered water, 0.9% saline, 0.3% glycine, hyaluronic acid, and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

In an advantageous embodiment, the cancer therapeutic is an immunotherapeutic (e.g., an antibody, such as pembrolizumab). The immunotherapeutic may be a cytokine therapeutic (such as an interferon or an interleukin), a dendritic cell therapeutic or an antibody therapeutic, such as a monoclonal antibody. In a particularly advantageous embodiment, the immunotherapeutic is a neoantigen (see, e.g., U.S. Pat. No. 9,115,402 and US Patent Publication Nos. 20110293637, 20160008447, 20160101170, 20160331822 and 20160339090).

In an advantageous embodiment, treatments for cancer caused by MSI mutations are contemplated. In particular, if the cancer caused by one or more MSI mutations overexpresses a programmed cell death protein 1 (PD-1) receptor ligand, a PD-1 receptor ligand inhibitor may be contemplated as a treatment. Advantageously, the PD-1 receptor ligand is PD-L1. One example of a PD-L1 receptor ligand inhibitor is pembrolizumab (formerly MK-3475 and lambrolizumab, trade name Keytruda) which is known to be efficacious for MSI cancers. In embodiments, the MSI cancer is unresectable or metastatic and/or associated with mismatch repair deficient (dMMR) or microsatellite instabilityphigh (MSI-H) solid tumors that have progressed following prior treatment. The present disclosure encompasses identifying whether a patient has an MSI cancer in order to know whether pembrolizumab would be a good choice for treatment for that patient, e.g., as a companion diagnostic. In particular, identifying of one or more of the mutations in a patient.

Without being bound by theory, pembrolizumab is a therapeutic antibody that binds to and blocks the PD-1, programmed cell death protein 1 located on lymphocytes. This receptor is generally responsible for preventing the immune system from attacking the body's own tissues; by acting as an immune checkpoint. Many cancers make proteins that bind to PD-1, thus shutting down the ability of the body to kill the cancer on its own. Inhibiting PD-1 on the lymphocytes prevents this, allowing the immune system to target and destroy cancer cells. Tumors that have mutations that cause DNA mismatch repair, which often results in microsatellite instability, tend to generate many mutated proteins that could serve as tumor antigens; pembrolizumab appears to facilitate clearance of any such tumor by the immune system, by preventing the self-checkpoint system from blocking the clearance.

The Food and Drug Administration (FDA) approved pembrolizumab on May 23, 2017, for the treatment of adult and pediatric patients with unresectable or metastatic, microsatellite instability-high (MSI-H) or mismatch repair deficient (dMMR) solid tumors that have progressed following prior treatment and who have no satisfactory alternative treatment options and for the treatment of unresectable or metastatic MSI-H or dMMR colorectal cancer that has progressed following treatment with a fluoropyrimidine, oxaliplatin, and irinotecan.

In particular, treatments for adrenal, breast, cervical, colon, endometrial, rectal or stomach cancer are especially contemplated.

For adrenal cancer, surgery is recommended to remove the entire adrenal gland. Standard treatment options for adrenocortical carcinoma (ACC) include, but are not limited to, chemotherapy with mitotane, chemotherapy with mitotane plus streptozotocin or mitotane plus etoposide, doxorubicin, and cisplatin, radiation therapy to bone metastases and/or surgical removal of localized metastases, particularly those that are functioning.

For breast cancer, local therapies such as surgery and radiation are recommended. Breast cancer may also be treated systemically by chemotherapy, hormone therapy (such as, but not limited to, tamoxifen, toremifene, fulvestrant or aromatase inhibitors) or targeted therapy (such as, but not limited to, monoclonal antibodies or other therapeutics that target a HER2 protein, a mTor protein or cyclin-dependent kinases, or kinase inhibitors). If the breast cancer is a BRCA cancer, the cancer may be treated and/or prevented by a mastectomy, sapingo-oophorectomy or hormonal therapy medicines, such as selective estrogen receptor modulators or aromatase inhibitors. Hormonal therapy medicines include, but are not limited to, tamoxifen, raloxifene, exemestane or anastrozole.

Cervical cancer may be treated by surgery, radiation, chemotherapy or targeted therapy (such as an angiogenesis inhibitor). Cervical squamous cell carcinoma may be treated by cryosurgery, laser surgery, loop electrosurgical excision procedure (LEEP/LEETZ), cold knife conization or a simple hysterectomy (as the first treatment or if the cancer returns after other treatments). Endocervical adenocarcinoma (CESC) may be treated by surgery or radiation.

Colon cancer may be treated by surgery or chemotherapy. Some common regimens for treating colon cancer include, but are not limited to: OLFOX: leucovorin, 5-FU, and oxaliplatin (Eloxatin); FOLFIRI: leucovorin, 5-FU, and irinotecan (Camptosar); CapeOX: capecitabine (Xeloda) and oxaliplatin; FOLFOXIRI: leucovorin, 5-FU, oxaliplatin, and irinotecan; One of the above combinations plus either a drug that targets VEGF (bevacizumab [Avastin], ziv-aflibercept [Zaltrap], or ramucirumab [Cyramza]), or a drug that targets EGFR (cetuximab [Erbitux] or panitumumab [Vectibix]); 5-FU and leucovorin, with or without a targeted drug; Capecitabine, with or without a targeted drug; Irinotecan, with or without a targeted drug; Cetuximab alone; Panitumumab alone; Regorafenib (Stivarga) alone; and/or Trifluridine and tipiracil (Lonsurf).

Endometrial cancer may be treated by surgery, chemotherapy, and radiation. Uterine corpus endometrial carcinoma (UCEC) is the most common type of endometrial cancer. Operative procedures used for managing endometrial cancer include the following: exploratory laparotomy, total abdominal hysterectomy, bilateral salpingo-oophorectomy, peritoneal cytology, and pelvic and para-aortic lymphadenectomy. Chemotherapeutic medications such as cisplatin can be used in the management of endometrial carcinoma. Standard treatment options for uterine carcinosarcoma (UCS) include surgery (total abdominal hysterectomy, bilateral salpingo-oophorectomy, and pelvic and periaortic selective lymphadenectomy), surgery plus pelvic radiation therapy, surgery plus adjuvant chemotherapy or surgery plus adjuvant radiation therapy (EORTC-55874).

Rectal cancer may be treated by surgery, chemotherapy, and radiation. Some common regimens for treating rectal cancer include, but are not limited to: FOLFOX: leucovorin, 5-FU, and oxaliplatin (Eloxatin); FOLFIRI: leucovorin, 5-FU, and irinotecan (Camptosar); CapeOX: capecitabine (Xeloda) and oxaliplatin; FOLFOXIRI: leucovorin, 5-FU, oxaliplatin, and irinotecan; One of the above combinations, plus either a drug that targets VEGF (bevacizumab [Avastin], ziv-aflibercept [Zaltrap], or ramucirumab [Cyramza]), or a drug that targets EGFR (cetuximab [Erbitux] or panitumumab [Vectibix]); 5-FU and leucovorin, with or without a targeted drug; Capecitabine, with or without a targeted drug; Irinotecan, with or without a targeted drug; Cetuximab alone; Panitumumab alone; Regorafenib (Stivarga) alone; and/or Trifluridine and tipiracil (Lonsurf).

Stomach cancer may be treated by surgery, radiation, chemotherapy, or targeted therapy (such as a monoclonal antibody or other therapeutics that target a HER2 protein or a VEGF receptor). Drugs approved for stomach cancer include, but are not limited to, Capecitabine (Xeloda). Cisplatin (Platinol), Cyramza (Ramucirumab), Docetaxel, Doxorubicin Hydrochloride, 5-FU (Fluorouracil Injection), Fluorouracil Injection, Herceptin (Trastuzumab), Irinotecan Hydrochloride, Leucovorin Calcium, Mitomycin C, Mitozytrex (Mitomycin C), Mutamycin (Mitomycin C), Ramucirumab, Taxotere (Docetaxel) and Trastuzumab and may be administered individually or in a combination thereof.

The therapeutics of the present disclosure may be delivered in a particle and/or nanoparticle delivery system. Several types of particle and nanoparticle delivery systems and/or formulations are known to be useful in a diverse spectrum of biomedical applications; and particle and nanoparticle delivery systems in the practice of the instant disclosure can be as in WO 2014/093622 (PCT/US13/74667). In general, a particle is defined as a small object that behaves as a whole unit with respect to its transport and properties. Particles are further classified according to diameter. Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm. As used herein, a particle delivery system/formulation is defined as any biological delivery system/formulation which includes a particle in accordance with the present disclosure. A particle in accordance with the present disclosure is any entity having a greatest dimension (e.g. diameter) of less than 100 microns (μm). In some embodiments, inventive particles have a greatest dimension of less than 10 μm. In some embodiments, inventive particles have a greatest dimension of less than 2000 nanometers (nm). In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 500 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the disclosure. In some embodiments, inventive particles have a greatest dimension ranging between 25 nm and 200 nm. Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarisation interferometry and nuclear magnetic resonance (NMR). Characterization (dimension measurements) may be made as to native particles (i.e., preloading) or after loading of the cargo to provide particles of an optimal size for delivery for any in vitro, ex vivo and/or in vivo application of the present disclosure. In certain preferred embodiments, particle dimension (e.g., diameter) characterization is based on measurements using dynamic laser scattering (DLS). Particles delivery systems within the scope of the present disclosure may be provided in any form, including but not limited to solid, semi-solid, emulsion, or colloidal particles. As such any of the delivery systems described herein, including but not limited to, e.g., lipid-based systems, liposomes, micelles, microvesicles, exosomes, or gene gun may be provided as particle delivery systems within the scope of the present disclosure.

In general, a “nanoparticle” refers to any particle having a diameter of less than 1000 nm. In certain preferred embodiments, nanoparticles of the disclosure have a greatest dimension (e.g., diameter) of 500 nm or less. In other preferred embodiments, nanoparticles of the disclosure have a greatest dimension ranging between 25 nm and 200 nm. In other preferred embodiments, nanoparticles of the disclosure have a greatest dimension of 100 nm or less. In other preferred embodiments, nanoparticles of the disclosure have a greatest dimension ranging between 35 nm and 60 nm. Nanoparticles encompassed in the present disclosure may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core—shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present disclosure.

Semi-solid and soft nanoparticles have been manufactured and are within the scope of the present disclosure. A prototype nanoparticle of semi-solid nature is the liposome. Various types of liposome nanoparticles are currently used clinically as delivery systems for anticancer drugs and vaccines. Nanoparticles with one half hydrophilic and the other half hydrophobic are termed Janus particles and are particularly effective for stabilizing emulsions. They can self-assemble at water/oil interfaces and act as solid surfactants. Doses of about 5 mg/kg are contemplated, with single or multiple doses, depending on the target tissue. It is mentioned herein experiments involving mice involve 20 g mammals and that dosing can be scaled up to a 70 kg human. With regard to nanoparticles that can deliver RNA, see, e.g., Alabi et al., Proc Natl Acad Sci USA. 2013 Aug. 6; 110(32):12881-6; Zhang et al., Adv Mater. 2013 Sep. 6; 25(33):4641-5; Jiang et al., Nano Lett. 2013 Mar. 13; 13(3):1059-64; Karagiannis et al., ACS Nano. 2012 Oct. 23; 6(10):8484-7; Whitehead et al., ACS Nano. 2012 Aug. 28; 6(8):6922-9 and Lee et al., Nat Nanotechnol. 2012 Jun. 3; 7(6):389-93. Lipid Nanoparticles, Spherical Nucleic Acid (SNA™) constructs, nanoplexes and other nanoparticles (particularly gold nanoparticles) are also contemplate as a means for delivery A recent publication, entitled “In vivo endothelial siRNA delivery using polymeric nanoparticles with low molecular weight” by James E. Dahlman and Carmen Barnes et al. Nature Nanotechnology (2014) published online 11 May 2014, doi:10.1038/nnano.2014.84, incorporated herein in its entirety, showed that polymeric nanoparticles made of low-molecular-weight polyamines and lipids can deliver siRNA to endothelial cells with high efficiency, thereby facilitating the simultaneous silencing of multiple endothelial genes in vivo. The authors reported that unlike lipid or lipid-like nanoparticles, the nanoparticle formulation they used (termed 7C1), differed from traditional lipid-based nanoparticle formulations because it can deliver siRNA to lung endothelial cells at low doses without substantially reducing gene expression in pulmonary immune cells, hepatocytes or peritoneal immune cells.

### Colorectal Cancer (CRC)

CRC is the third most common cancer type in which about 1.4 million new cases are diagnosed each year. Additionally, CRC results in about 700,000 deaths per year. Unfortunately, the frequency of CRC appears to be increasing throughout the developed world, presumably due to increased risk of CRC associated with alcohol consumption, smoking, obesity, diabetes, the consumption of large amounts of meat, and little physical activity.

About 15% are associated with microsatellite instability (MSI), which can be defined as somatic changes in the length of microsatellites. Based on microsatellite status (e.g., MSI versus MSS), colorectal tumors can be divided into 3 the categories: 1. tumors with high levels of microsatellite instability (MSI-H), 2 tumors with low levels of microsatellite instability (MSI-L), and tumors that are microsatellite stable (MSS).

Lynch syndrome is a hereditary form of autosomal dominant colon cancer that results from inherited mismatch repair gene defects and is characterized by high levels of microsatellite instability and constitutes about 20% of MSI-H CRCs. Lynch Syndrome patients typically display initial cancer onset in their mid-forties, which is in sharp contrast to patients with sporadic MSI-H cancers where the average age is over seventy.

Sporadic MSI-H tumors are usually caused by the epigenetic silencing of MLH1 caused by promoter methylation. Traditionally, Lynch Syndrome tumors are thought to arise from adenomas, while sporadic MSI-H CRCs are believed to arise from serrated polyps. Approximately 80% of MSI-H tumors are sporadic tumors. Sporadic MSI-H tumors are generally predisposed to present in the proximal colon and are more common in women than men.

With respect to CRC, it is therefore clear that the ability to accurately assess MSI status is important because it can define hereditary forms of CRC and inform clinical care. Additionally, identifying patients with Lynch Syndrome is important because they and their relatives have a high risk of developing second primary cancers. Early detection of these cancers has a significant impact upon prognosis, and it has been estimated that more than 60% of Lynch Syndrome cancer deaths could be prevented with proper follow up.

Other exemplary MSI cancers include, but are not limited to, adenocarcinoma (COAD), stomach adenocarcinoma (STAD), and uterine corpus endometrial carcinoma (UCEC).

### MSI Classification

The methods and compositions described herein relate to identification of a new and clinically useful classifier for MSI, the development of which is based upon an assessment of low pass (e.g., about 0.01×) WGS data for a neoplasia or tumor sample. Specific components of the instant MSI classifier include the following.

In embodiments, the methods of the present disclosure involve calculating an MSI-Score as described herein. In some instances, the methods involve classifying a biological sample as containing microsatellite instability if the MSI-Score is above a threshold. In embodiments, the threshold is about or at least about log 10(1), log 10(5), log 10(10), log 10(15), log 10(20), log 10(25), log 10(50), log 10(100), or log 10(1000).

### Reference Sequences

In certain aspects, the instant disclosure provides methods and kits that involve and/or allow for assessment of the presence or absence of one or more sequence variants and/or mutations in a test subject, tissue, cell or sample, as compared to a corresponding reference sequence. In particular embodiments, a subject, tissue, cell and/or sample is assessed for one or more variants and/or sites of copy number variation within the sequences/sequence locations (e.g., motif A as defined below).

### Amplification and Sequencing Oligonucleotides

In some aspects, WGS or exome sequencing may be performed upon a test sample for purpose of detecting variants and/or copy number variation as described herein and identifying MSI classification and selecting a therapy. In certain embodiments, assessment of candidate and/or test MSI neoplasia or tumor samples can be performed using one or more amplification and/or sequencing oligonucleotides flanking the above-referenced variant sequence and/or copy number variation regions. Design and use of such amplification and sequencing oligonucleotides, and/or copy number detection probes/oligonucleotides, can be performed by one of ordinary skill in the art.

As will be appreciated by one of ordinary skill in the art, any such amplification sequencing and/or copy number detection oligonucleotides can be modified by any of a number of art-recognized moieties and/or exogenous sequences, e.g., to enhance the processes of amplification, sequencing reactions and/or detection. Exemplary oligonucleotide modifications that are expressly contemplated for use with the oligonucleotides of the instant disclosure include, e.g., fluorescent and/or radioactive label modifications; labeling one or more oligonucleotides with a universal amplification sequence (optionally of exogenous origin) and/or labeling one or more oligonucleotides of the instant disclosure with a unique identification sequence (e.g., a “bar-code” sequence, optionally of exogenous origin), as well as other modifications known in the art and suitable for use with oligonucleotides.

### Neural Network Classification

In certain exemplified aspects, a neural network classifier may also be used may be used to define MSI classification groups. As would be appreciated by one of ordinary skill in the art, other forms of classifier (e.g., nearest-neighbor and various others) can be applied to variant and/or copy number data, to perform such test sample classification.

A neural network consists of units (neurons), arranged in layers, which convert an input vector into some output. Each unit takes an input, applies a function (e.g., a nonlinear function) to it and then passes the output on to the next layer. Generally the networks are defined to be feed-forward: a unit feeds its output to all the units on the next layer, but there is no feedback to the previous layer. Weightings are applied to the signals passing from one unit to another, and it is these weightings which are tuned in the training phase to adapt a neural network to the particular problem at hand. This is the learning phase.

Neural networks have found application in a wide variety of problems. These range from function representation to pattern recognition, with pattern recognition being the focus of use of neural net classifiers of the instant disclosure.

### Clinical Classifier Scoring Algorithm

The techniques herein provide a classifier algorithm to identify neoplasia or tumor samples as either MSI or MSS. The classifier algorithm herein is based, in part, on using high throughput NGS systems to generate sequencing data for as many loci as possible within the neoplasia or tumor, aggregating the WGS data, and applying a weighting system for analysis. For example, if a particular MS locus has 11-15 repeats of the A motif, it may receive a weight score of 1; however, if that particular MS locus does not have 11-15 repeats of the A motif, it will receive a weight score of 0. In this regard, the techniques herein allow generation of indel signature patterns characteristic of either MSI or MSS.

Without being bound by theory, the techniques herein have identified approximately 600,000 loci having 11-15 repeats of the A motif, which means that even if the WGS data for a particular neoplasia or tumor sample has a very low pass coverage of the genome (e.g., 90%-95% of the loci are not covered at all), it will still be sufficient to accurately identify an MSI indel signature pattern and be able to assess the neoplasia or tumor sample as being either MSI or MSS.

It is expressly contemplated that a classifier of the instant disclosure can be used to link discrete genetic signatures, clinical outcome and specific targeted therapy in clinical trials and in practice. Specifically, it is contemplated that neoplasia or tumors of patients with MSI can be analyzed prospectively with an exemplified classifier or other classifier within the scope of the instant disclosure. The resulting cluster identifications are predictive of the likelihood of response to standard combination chemotherapy and suggest rational targeted therapies based on cluster-specific biology. Additionally, the resulting identifications can determine whether or not a patient is eligible for anti-PDL or anti-PDL1 treatment. It is further expressly contemplated that a classifier of the instant disclosure can also be applied retrospectively to archival tissue from patients on specific clinical trials or therapies.

### Treatment Selection

The methods described herein can be used for selecting, and then optionally administering, an optimal treatment for a subject. Thus the methods described herein include methods for the treatment of cancer, particularly neoplasia or tumors associated with MSI. Generally, the methods include administering a therapeutically effective amount of a treatment as described herein, to a subject who is in need of, or who has been determined to be in need of, such treatment.

As used in this context, to “treat” means to ameliorate at least one symptom of the cancer. For example, a treatment can result in a reduction in tumor size, tumor growth, cancer cell number, cancer cell growth, or metastasis or risk of metastasis.

For example, the methods can include selecting and/or administering a treatment that includes a therapeutically effective amount of an immune checkpoint blocker such as, for example, cytotoxic T-lymphocyte antigen-4 (CTLA-4) and programmed death-1 (PD-1), to a subject having a select MSI tumor or cancer/tumor. In an embodiment, the immune checkpoint blocker is pembrolizumab.

Therapeutic agents specifically implicated for administration in using the instant MSI classifier include inhibitors of the following genetic targets: PD-1 and PD-L1.

### PD-1

The PD-1 receptor-ligand interaction is a major pathway hijacked by tumors to suppress immune control. PD-1, which is expressed on the cell surface of activated T-cells under healthy conditions, normally functions to down-modulate unwanted or excessive immune responses, including autoimmune reactions. The ligands for PD-1 (PD-L1 and PD-L2) are constitutively expressed or can be induced in various tumors. Binding of either PD-L1 or PD-L2 to PD-1 inhibits T-cell activation triggered through the T-cell receptor.

PD-L1 is expressed at low levels on various non-hematopoietic tissues, most notably on vascular endothelium, whereas PD-L2 protein is only detectably expressed on antigen-presenting cells found in lymphoid tissue or chronic inflammatory environments. PD-L2 is thought to control immune T-cell activation in lymphoid organs, whereas PD-L1 serves to dampen unwarranted T-cell function in peripheral tissues. Although healthy organs express little (if any) PD-L1, a variety of cancers were demonstrated to express abundant levels of this T-cell inhibitor. High expression of PD-L1 on tumor cells (and to a lesser extent of PD-L2) has been found to correlate with poor prognosis and survival in various cancer types, including renal cell carcinoma (RCC), pancreatic carcinoma, hepatocellular carcinoma, ovarian carcinoma and non-small cell lung cancer (NSCLC). Furthermore, PD-1 has been suggested to regulate tumor-specific T cell expansion in patients with malignant MEL. The observed correlation of clinical prognosis with PD-L1 expression in multiple cancers suggests that the PD-1/PD-L1 pathway plays a role in tumor immune evasion and should be considered as an attractive target for therapeutic intervention.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Combination Treatments

The compositions and methods of the present disclosure may be used in the context of a number of therapeutic or prophylactic applications. In order to increase the effectiveness of a treatment with the compositions of the present disclosure, e.g., a PD-1/PD-L1 inhibitor selected and/or administered as a single agent, or to augment the protection of another therapy (second therapy), it may be desirable to combine these compositions and methods with one another, or with other agents and methods effective in the treatment, amelioration, or prevention of diseases and pathologic conditions, for example, neoplasia or tumors identified as MSI.

Administration of a composition of the present disclosure to a subject will follow general protocols for the administration described herein, and the general protocols for the administration of a particular secondary therapy will also be followed, taking into account the toxicity, if any, of the treatment. It is expected that the treatment cycles would be repeated as necessary. It also is contemplated that various standard therapies may be applied in combination with the described therapies.

### Pharmaceutical Compositions

Agents of the present disclosure can be incorporated into a variety of formulations for therapeutic use (e.g., by administration) or in the manufacture of a medicament (e.g., for treating or preventing a MSI tumor or cancer with, for example, PD-1/PD-L1 inhibitors, such as pembrolizumab) by combining the agents with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols.

For example, MSI neoplasia or tumors described herein may be treated with therapeutic agents such as, for example, immunotherapeutic agents that act by effectively stimulating the immune response, e.g., PD-1/PD-L1 inhibitors (e.g., Pembrolizumab). Pembrolizumab is a humanized monoclonal antibody that blocks the interaction between PD-1 and its ligands, PD-L1 and PD-L2. Pembrolizumab is an IgG4 kappa immunoglobulin with an approximate molecular weight of 149 kDa. Pembrolizumab is believed to have a mechanism of action in which binding of the PD-1 ligands, PD-L1 and PD-L2, to the PD-1 receptor found on T cells, inhibits T cell proliferation and cytokine production. Upregulation of PD-1 ligands occurs in some tumors and signaling through this pathway can contribute to inhibition of active T-cell immune surveillance of tumors. Pembrolizumab binds to the PD-1 receptor and blocks its interaction with PD-L1 and PD-L2, releasing PD-1 pathway-mediated inhibition of the immune response, including the anti-tumor immune response. In syngeneic mouse tumor models, blocking PD-1 activity resulted in decreased tumor growth.

Programmed cell death 1 (PD-1) and programmed death ligand 1 (PD-L1) blockade as a potential form of cancer immunotherapy are based on the fact that activation of the PD-1/PD-L1 axis serves as a mechanism for tumor evasion of host tumor antigen-specific T-cell immunity. Accordingly, inhibition of PD-1/PDL-1 interaction (and corresponding downstream signaling events) strengthen tumor antigen-specific T-cell responses and corresponding tumor antigen-specific T-cell immunity. Other FDA approved PD-1/PD-L1 immunotherapeutic inhibitors include Nivolumab, which like Pembrolizumab, is a PD-1 inhibitor antibody, and Atezolizumab, Durvalumab, and Avelumab, which are all PD-L1 inhibitor antibodies.

In addition to immunotherapeutic treatments, the invention includes treatment with additional agents, either alone or in combination with the immunotherapeutic treatment (such as the anti-PD-1/PDL-1 therapeutic agent). Examples of such agents include chemotherapeutic agents including chemotherapeutic alkylating agents such as Cyclophosphamide, Mechlorethamine, Chlorambucil, Melphalan, Monofunctional alkylators, Dacarbazine, nitrosoureas, and Temozolomide (Oral dacarbazine); anthracyclines such as Daunorubicin, Doxorubicin, Epirubicin, Idarubicin, Mitoxantrone, Valrubicin, cytoskeletal disruptor agents (taxanes) such as Paclitaxel, Docetaxel, Abraxane and Taxotere; Epothilones; Histone deacetylase inhibitors such as Vorinostat and Romidepsin; topoisomerase I inhibitors such as Irinotecan and Topotecan; topoisomerase II inhibitors such as Etoposide, Teniposide, and Tafluposide; Kinase inhibitors such as Bortezomib, Erlotinib, Gefitinib, Imatinib, Vemurafenib, and Vismodegib; nucleotide analogs and precursor analog agents such as Azacitidine, Azathioprine, Capecitabine, Cytarabine, Doxifluridine, Fluorouracil, Gemcitabine, Hydroxyurea, Mercaptopurine, Methotrexate, and Tioguanine (formerly Thioguanine); peptide antibiotics such as Bleomycin and Actinomycin; Platinum-based agents such as Carboplatin, Cisplatin, Oxaliplatin; Retinoids such as Retinoids, Tretinoin, Alitretinoin, Bexarotene; Vinca alkaloids and derivatives such as Vinblastine, Vincristine, Vindesine and Vinorelbine; as well as other chemotherapeutic agents including all-trans retinoic acid, Docetaxel, Doxifluridine, Epothilone, Fluorouracil, Methotrexate, and Pemetrexed.

A chemotherapeutic agents drugs for use with the invention include any chemical compound used in the treatment of a proliferative disorder. Chemotherapeutic agents include, but are not limited to, RAF inhibitors (e.g., BRAF inhibitors), MEK inhibitors, PI3K inhibitors and AKT inhibitors. Other chemotherapeutic agents include, without being limited to, the following classes of agents: nitrogen mustards, e.g., cyclophosphamide, trofosfamide, ifosfamide and chlorambucil; nitroso ureas, e.g., carmustine (BCNU), lomustine (CCNU), semustine (methyl CCNU) and nimustine (ACNU); ethylene imines and methyl-melamines, e.g., thiotepa; folic acid analogs, e.g., methotrexate; pyrimidine analogs, e.g., 5-fluorouracil and cytarabine; purine analogs, e.g., mercaptopurine and azathioprine; vinca alkaloids, e.g., vinblastine, vincristine and vindesine; epipodophyllotoxins, e.g., etoposide and teniposide; antibiotics, e.g., dactinomycin, daunorubicin, doxorubicin, epirubicin, bleomycin a2, mitomycin c and mitoxantrone; estrogens, e.g., diethyl stilbestrol; gonadotropin-releasing hormone analogs, e.g., leuprolide, buserelin and goserelin; antiestrogens, e.g., tamoxifen and aminoglutethimide; androgens, e.g., testolactone and drostanolonproprionate; platinates, e.g., cisplatin and carboplatin; and interferons, including interferon-alpha, beta and gamma.

Chemotherapeutic agents include, for example, RAF inhibitors (e.g. Vemurafenib or Dabrafenib), MEK inhibitors, PI3K inhibitors, or AKT inhibitors. The RAF inhibitor is, for example, a BRAF inhibitor. The chemotherapeutic agents can be administered alone or in combination (e.g., RAF inhibitors with MEK inhibitors). The cancer is any cancer in which the tumor has a B-RAF activating mutation. For example the cancer is melanoma, colon cancer, lung cancer, brain cancer, hematologic cancers or thyroid cancer.

In addition, these modulatory agents can also be administered in combination therapy with, e.g., chemotherapeutic agents, hormones, antiangiogens, radiolabeled, compounds, or with surgery, cryotherapy, and/or radiotherapy. The preceding treatment methods can be administered in conjunction with other forms of conventional therapy (e.g., standard-of-care treatments for cancer well known to the skilled artisan), either consecutively with, pre- or post-conventional therapy.

The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer (e.g., a hematological cancer, such as DLBCL), being treated, the combined use of immunotherapeutic agent (e.g., anti-PD1/PDL1), the extent of the disease and other factors familiar to the physician of skill in the art and can be determined by the physician.

Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further examples of formulations that are suitable for various types of administration can be found in Remington's Pharmaceutical Sciences, Mace Publishing Company, Philadelphia, PA, 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, e.g. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Pharmaceutical Dosages

Pharmaceutical compositions of the present disclosure containing an agent described herein may be used (e.g., administered to an individual, such as a human individual, in need of treatment with a PD-1/PD-L1 inhibitor, etc.) in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, or about 2 mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfate, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (0 absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent (e.g., a BCL2 inhibitor, PI3K inhibitor, BCR/TLR signaling inhibitor, JAK/STAT inhibitor, etc.) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

FDA-approved drugs provided herein are typically formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for topical administration to the eye of a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent (e.g., a PD-1/PD-L1 inhibitor, etc.) described herein.

As noted elsewhere herein, a drug of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

The FDA-approved drug or other therapy is administered to the subject in an amount sufficient to achieve a desired effect at a desired site (e.g., reduction of cancer size, cancer cell abundance, symptoms, etc.) determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., a PD-1/PD-L1 inhibitor, etc.) described herein. In certain embodiments, a dose described herein includes independently between 1 mg and 3 mg, inclusive, of an agent (e.g., a PD-1/PD-L1 inhibitor, etc.) described herein. In certain embodiments, a dose described herein includes independently between 3 mg and 10 mg, inclusive, of an agent (e.g., a PD-1/PD-L1 inhibitor, etc.) described herein. In certain embodiments, a dose described herein includes independently between 10 mg and 30 mg, inclusive, of an agent (e.g., a PD-1/PD-L1 inhibitor, etc.) described herein. In certain embodiments, a dose described herein includes independently between 30 mg and 100 mg, inclusive, of an agent (e.g., a PD-1/PD-L1 inhibitor, etc.) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g., a PD-1/PD-L1 inhibitor, etc.) or composition, as described herein, can be administered in combination with one or more additional pharmaceutical agents (e.g., therapeutically and/or prophylactically active agents), which are different from the agent or composition and may be useful as, e.g., combination therapies. The agents or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk of developing a disease in a subject in need thereof, in inhibiting the replication of a virus, in killing a virus, etc. in a subject or cell. In certain embodiments, a pharmaceutical composition described herein including an agent (e.g., a PD-1/PD-L1 inhibitor, etc.) described herein and an additional pharmaceutical agent shows a synergistic effect that is absent in a pharmaceutical composition including one of the agent and the additional pharmaceutical agent, but not both.

In some embodiments of the disclosure, a therapeutic agent distinct from a first therapeutic agent of the disclosure is administered prior to, in combination with, at the same time, or after administration of the agent of the disclosure. In some embodiments, the second therapeutic agent is selected from the group consisting of a chemotherapeutic, an antioxidant, an anti-inflammatory agent, an antimicrobial, a steroid, etc.

The agent or composition can be administered concurrently with, prior to, or subsequent to one or more additional pharmaceutical agents, which may be useful as, e.g., combination therapies. Pharmaceutical agents include therapeutically active agents.

Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, chemotherapeutic agents, other epigenetic modifier inhibitors, etc., other anti-cancer agents, immunomodulatory agents, anti-proliferative agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine kinase inhibitors), modulators of protein stability (e.g., proteasome inhibitors), Hsp90 inhibitors, glucocorticoids, all-trans retinoic acids, and other agents that promote differentiation. In certain embodiments, the agents described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Kits

The instant disclosure also provides kits containing agents of this disclosure for use in the methods of the present disclosure. Kits of the instant disclosure may include one or more containers comprising an agent (e.g., a PD-1/PD-L1 inhibitor, etc.) of this disclosure and/or may contain agents (e.g., oligonucleotide primers, probes, etc.) for identifying a cancer or subject as possessing one or more variant sequences. In some embodiments, the kits further include instructions for use in accordance with the methods of this disclosure. In some embodiments, these instructions comprise a description of administration of the agent to treat or diagnose, e.g., a neoplasia or tumor having MSI, according to any of the methods of this disclosure. In some embodiments, the instructions comprise a description of how to detect a MSI class of cancer, for example in an individual, in a tissue sample, or in a cell.

The instructions generally include information as to dosage, dosing schedule, and route of administration for the intended treatment. The containers may be unit doses, bulk packages (e.g., multi-dose packages) or sub-unit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, e.g., a class of MSI cancer, in a subject. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). In certain embodiments, at least one active agent in the composition is a PD-1/PD-L1 inhibitor, an epigenetic modifier, an epigenetic modifier inhibitor, etc. The container may further comprise a second pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: MSIDetect

Mismatch repair (MMR) deficiency is often acquired early in tumorigenesis in a subset of cancers and is associated with an increase in the number of mutations, in particular of insertions and deletions (indels) in short repetitive DNA sequences called microsatellites (MSs). Microsatellite instability (MSI) is more frequent in colon, stomach, and endometrial cancers (˜10-20%), but also occurs with lower incidence in many other tumor types Recently, MSI cancers attracted attention due to their response to PD1/PD-L1 immune checkpoint blockade therapies that are now FDA approved for all MSI solid tumors. Currently, MSI is classified using protein or DNA obtained from tumor tissue biopsies. An optional alternative and much less-invasive approach is to obtain tumor DNA from cell free DNA (cfDNA) that is shed to the blood from tumor cells. Detecting MSI tumors using cfDNA has multiple advantages, as it may enable: (i) MSI classification when a tissue biopsy does not exist or is not feasible to obtain; (ii) identifying and monitoring patients with minimal residual disease; and (iii) potentially early detection of MSI cancers. Early detection is particularly important for Lynch syndrome cases, a syndrome caused by germline MMR deficiency. These individuals have a 95% lifetime risk of developing MSI tumors, and thus need to be frequently monitored with onerous procedures, such as colonoscopies or endometrial biopsies.

Traditional methods for classifying MSI tumors were not developed for analyzing cfDNA from blood. One common MSI detection method is based on immunohistochemistry that measures protein expression levels of the MMR proteins (MLH1, MSH2, MSH6 and PMS2) on tumor histology slides. Tumor samples without expression of any of these proteins are classified as MSI. Another common approach is based on gel electrophoresis of DNA from normal-matched tumors that captures indels at 7 specific frequently mutated microsatellite loci (the Bethesda protocol). Tumors with somatic indels in more than 2 of the 7 loci are classified as MSI. However, this method also suffers from a lack of sensitivity in samples with low purity where it is not possible to differentiate between a real indel and experimental noise.

A few, next-generation sequencing (NGS) methods that analyze tissue biopsies have been developed. These methods are typically based on identifying MS-indels in the MS loci in the sequenced region. These methods were adapted for panel sequencing data that sequence only a limited number of genes.

Recently, methods for detecting MSI tumors from cfDNA were proposed. These methods are based on extremely deep sequencing (20,000×) of ˜100 MS-loci, in addition to barcoding the DNA fragments to reduce the sequencing noise. These methods that combine very deep sequencing with DNA barcoding can detect MSI in cfDNA with as low as 0.1% cancer DNA.

MSIDetect is presented herein. MSIDetect is a new MSI detection method that does not detect indels at particular loci, but rather classifies the tumor as a whole by combining information from all the sequenced MS loci simultaneously. Therefore, while at very low cancer fractions the confidence that a particular MS locus is mutated is low, the aggregated evidence accumulated across all MS loci can be used to detect MSI cancers with high accuracy. MSIDetect does not require deep sequencing and can be applied to whole-genome sequencing (WGS) at very low depth (e.g., less than 0.5×, 0.1×, or 0.01×coverage), thus dramatically reducing the assay's cost. In addition it does not require a matched normal sample that some other tools require. Even WGS at ˜0.5×was sufficient for detecting MSI in cfDNA with >0.1% cancer DNA. Another advantage of using low-pass WGS is that, in addition to detecting MSI, it can be used for detection of copy-number alterations.

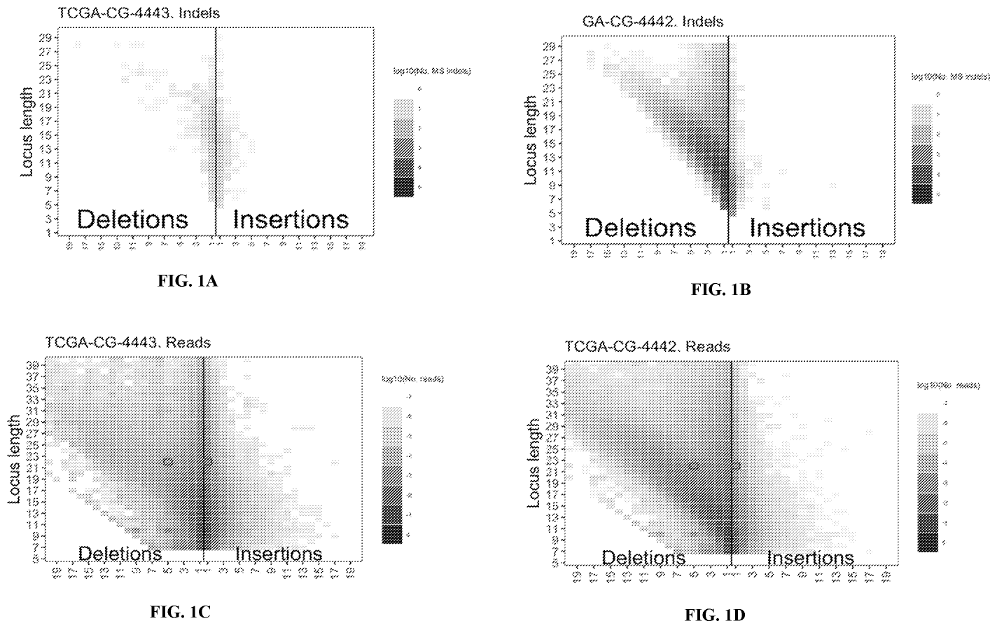

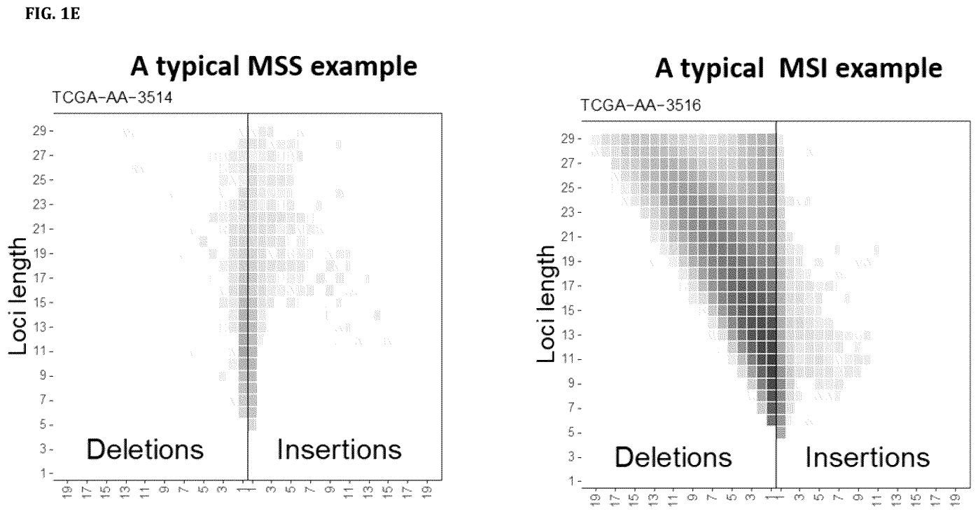

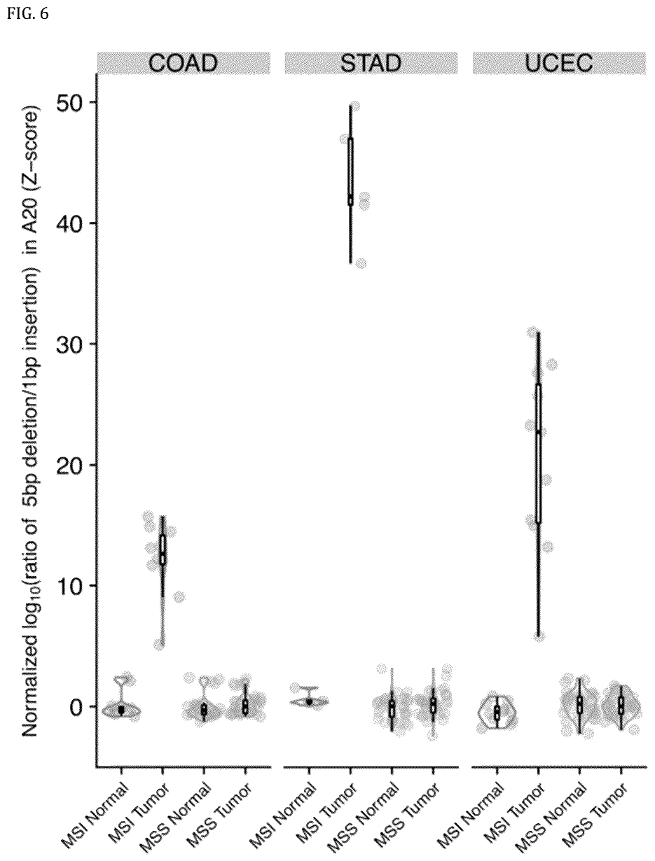

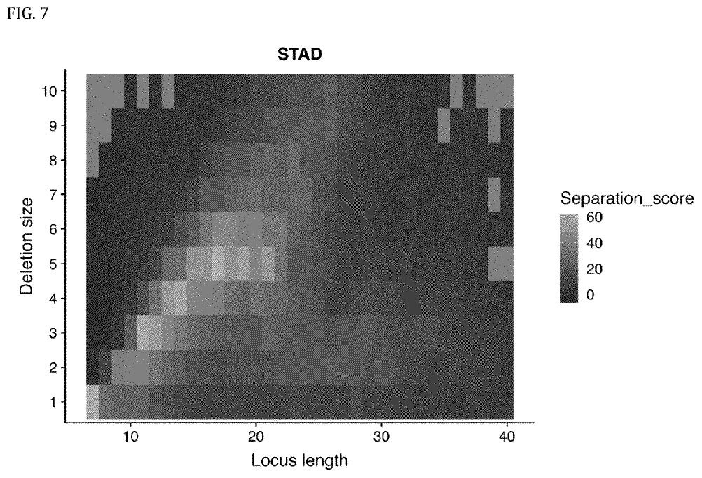

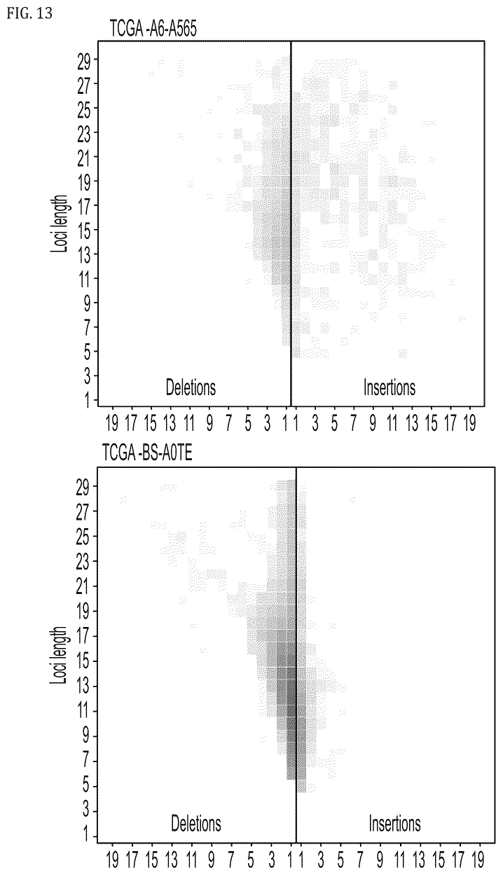

First, MSMuTect (Maruvka, Y., Mouw, K., Karlic, R. et al. Analysis of somatic microsatellite indels identifies driver events in human tumors. Nat Biotechnol 35, 951-959 (2017). doi.org/10.1038/nbt.3966) was optimized https://paperpile.com/c/uzVxt/WBItv for identifying somatic indels in the 23 million MS loci in the entire genome. MSMuTect was applied to 148 WGS from tumor-normal pairs, including 45 colon, 39 stomach and 51 endometrial cancers from The Cancer Genome Atlas (TCGA) project. As expected, the MSI tumors contained ˜100 times more MS-indels than the microsatellite stable (MSS) tumors (FIG. 5). Importantly, in addition to their increased MS-indel burden, the MSI tumors exhibited a different pattern of MS-indels. When comparing the ratio between insertions and deletions, MSI tumors had a strong bias towards deletions when compared to the MSS tumors [median fraction of deletions of 0.93 (3,924,179/4,234,486) in MSI vs. 0.52 (121,011/230,955) in MSS, P=10⁻¹⁶ fisher exact test (FIGS. 1A-1B, 1E, 6, 7, and 13). This bias was even stronger when considering indels greater than 2 bp (0.993 in the MSI and 0.32 in the MSS, fisher exact test <10⁻¹⁶; Methods).

Since there was such a profound difference between the types of indels in MSI and MSS tumors, a method was developed that does not require a matched normal sample and can detect MSI cancers with much lower sequencing depths. Instead of identifying MS-indels based on deep sequencing of each MS-locus and comparing the observed events between the tumor and its matched normal, indels could be identified in individual reads with respect to the reference genome. These differences from reference contain a mixture of sequencing errors, germline, and somatic events. However, even with these different sources of indels, the MSI and MSS patterns can be clearly identified, suggesting this difference can be used for MSI tumor detection (FIGS. 1C-D).

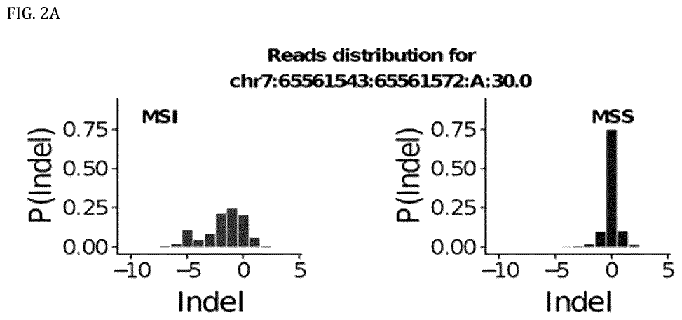

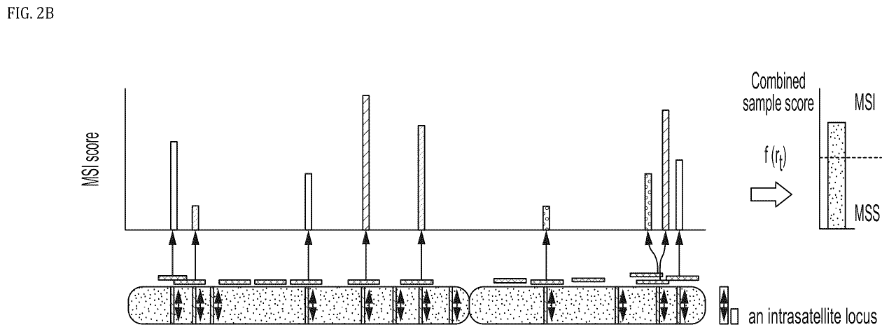

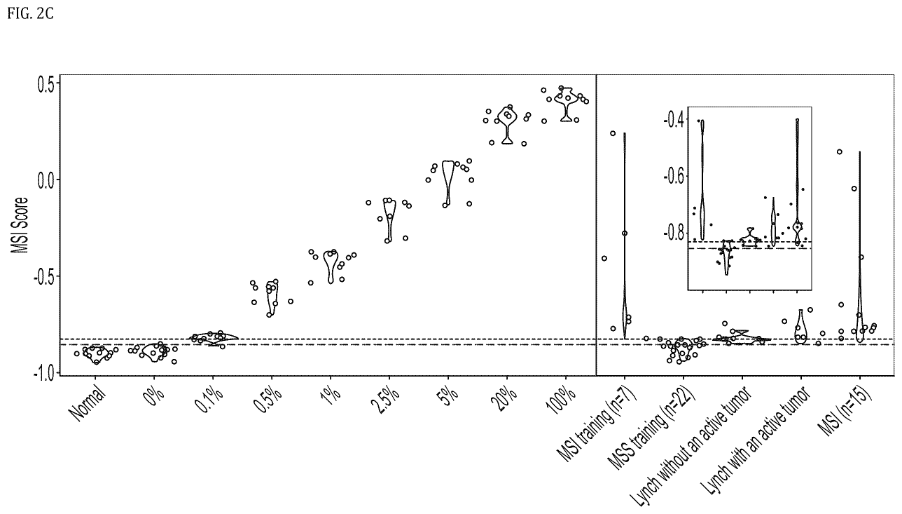

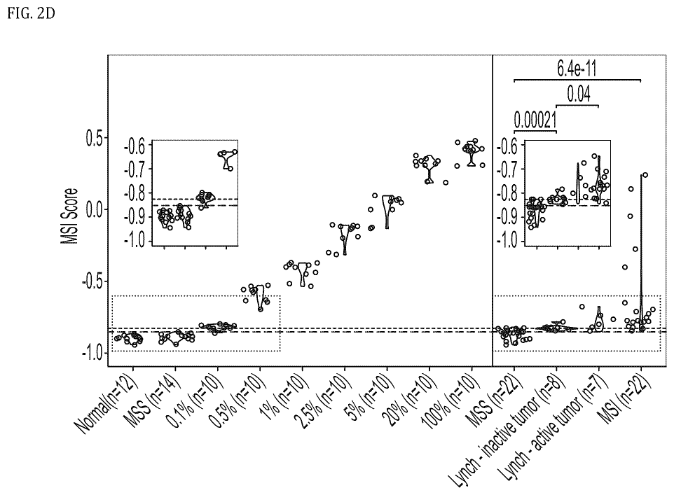

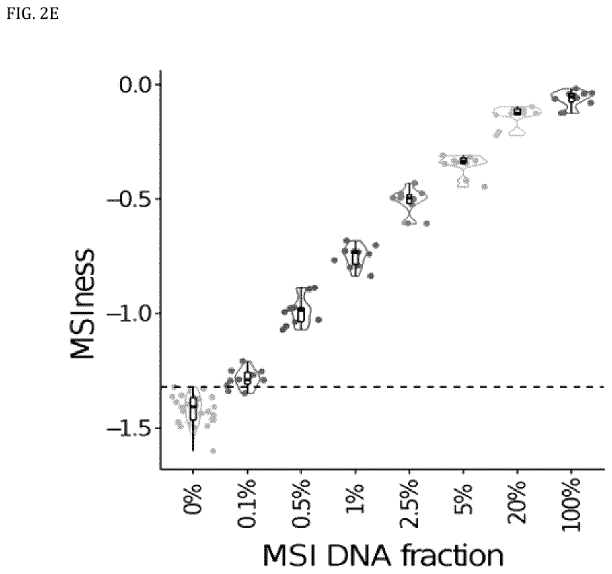

Using whole genome sequencing of 33 MSI cell-lines and 289 MSS cell-lines, from the CCLE, for each locus the number of reads that support different deviations from the reference were counted (FIG. 2A). Reads were counted and not events and the deviations from the reference did not necessarily reflect true MS-indels but also included sequencing errors at the locus. Next, these histograms were used as empirical likelihood functions. For example, if a read in the locus, depicted in FIG. 2A, had a 5 base deletion, it was more likely to be observed in an MSI case, while if it contained the reference number of repeats it was more likely to come from an MSS tumor.

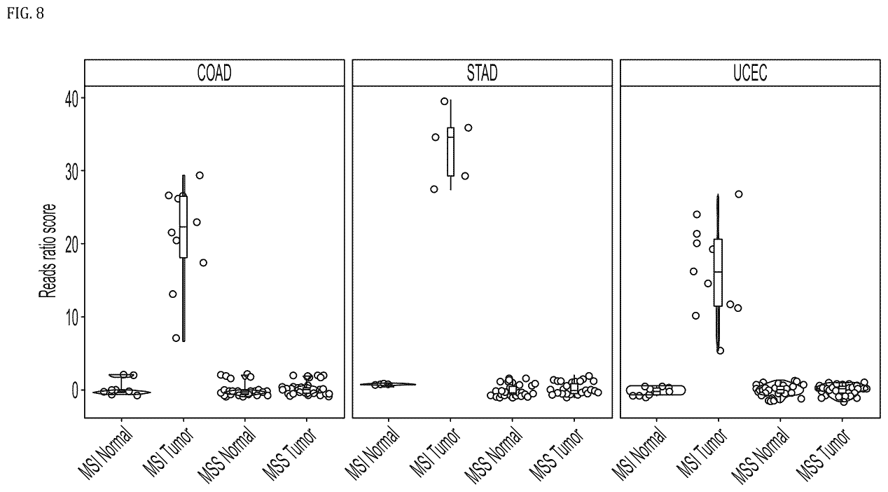

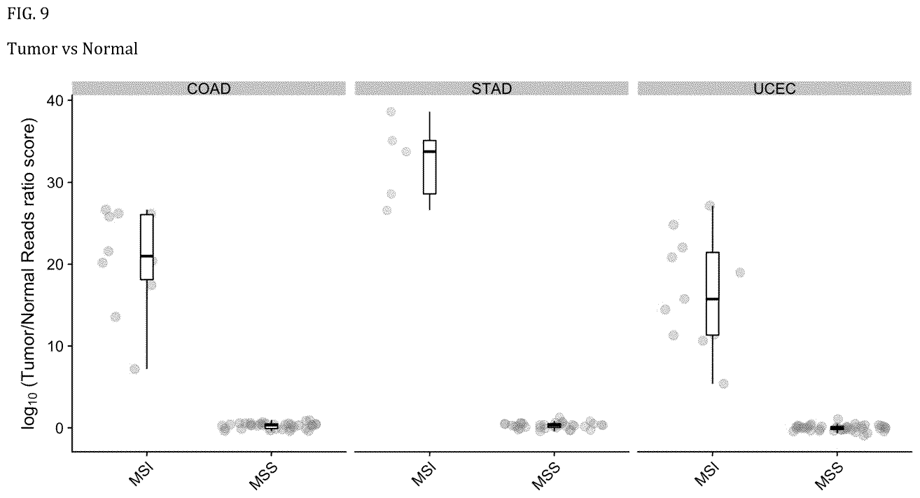

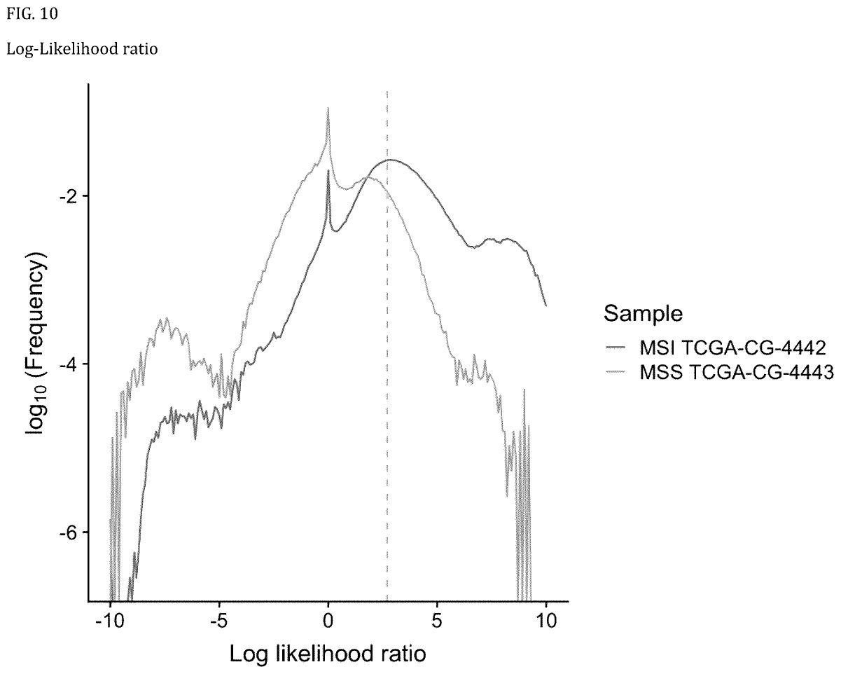

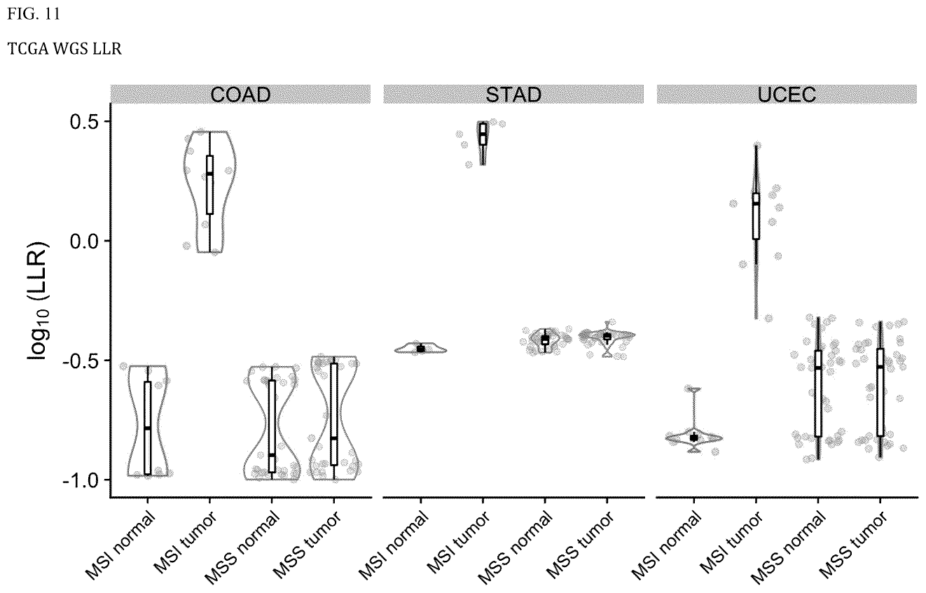

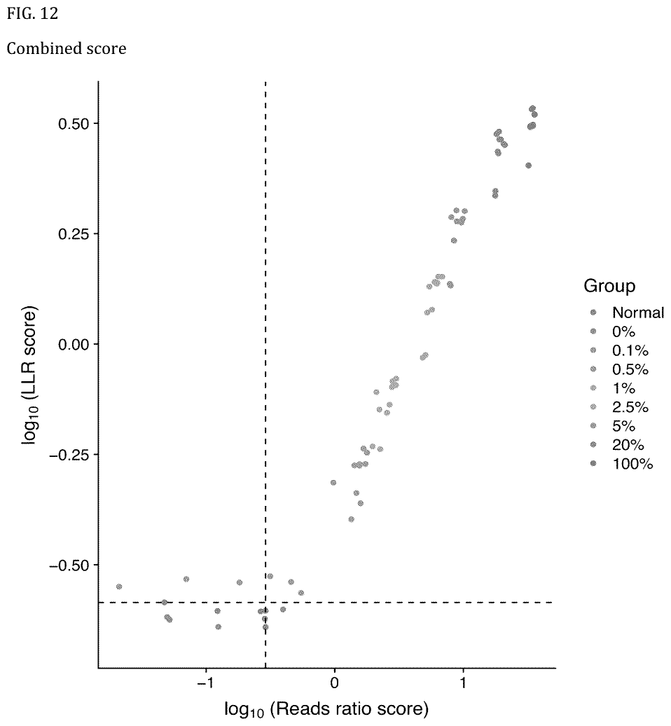

In order to classify samples as MSI vs. MSS, a score was calculated, termed the “MSI-score”, that is based on the log likelihood ratio (LLR) of each read coming from an MSI vs. an MSS tumor (FIGS. 2B, and 10-12). In particular, the MSI-score reflected the average LLR for reads that strongly support MSI vs. MSS. A reads ratio score (RR score=log₁₀(ratio of 5 bp deletions/1 bp insertions) was also calculated to distinguish between different tumor types (FIGS. 8, 9, 12).

Since the goal was to detect MSI cases from cell-free DNA, which often contains only a small fraction of tumor DNA, the performance of the MSI-score was evaluated in a wide range of DNA fractions (FIGS. 2C-2E). DNA was mixed from five different MSI cancer cell-lines (DV-90, Hec-108, Hec-59, Hec-6 and SNU-C2A) with normal DNA (NA12878) in 7 different fractions, between 0.1% to 100% (each fraction was duplicated; n=5×7×2=70). As controls DNA was alxo mixed from an MSS cancer cell-line (SK-N-MC) using the same fractions (n=7×2=14), the normal DNA was also used to represent 0% cancer (in 12 replicates). Low-pass whole-genome sequencing (coverage of −0.5×) was then used for these 96 DNA mixtures. It was found that the MSI-score is able to detect all the MSI cases in tumor fractions >0.1% and 8/10 of the 0.1% cases (FIGS. 2C-2E).

MSIDetect was applied to 59 cfDNA samples from colon cancer patients, including 22 MSS tumors, 22 sporadic MSI tumors, and 15 Lynch syndrome cases (7 with active cancer and 8 without). As in the DNA mixing experiments, the MSI-score in the cfDNA samples was significantly different between the MSS and MSI cases (FIGS. 2C, 2D, 10, and 11). Both Lynch syndrome groups were also significantly different than MSS, with a bigger difference for the Lynch cases with active cancer (Mann-Whitney U-test p<1.5×10⁻⁵; FIG. 2C). The Lynch syndrome cases with active cancer had similar MSI-scores to the sporadic MSI cases (p=0.16), with the exception of a few outlier high scores in the sporadic samples. Not intending to be bound by theory, this may reflect lower tumor DNA in the cfDNA or that the MSI-score is better tuned to detect sporadic MSI cases. Interestingly, also the Lynch syndrome cases with no active cancer had a slightly elevated MSI-score compared to the MSS cases (p=0.04), which may represent non-cancerous (e.g. polyps) or undetected lesions in these patients that shed DNA to the blood.

Setting a threshold based on the highest MSI-score among the 22 MSS cfDNA cases, MSI, 6/7 Lynch cases with active cancer, and 4/8 Lynch without active cancer exceed the threshold. Using iChorCNA to estimate the fraction of tumor DNA in the cfDNA, it was found that the MSI-score is more sensitive to detect MSI cancers in cell-free DNA. Not intending to be boundy by theory, two factors can contribute to the improved sensitivity of MSIDetect compared to iChorCNA: (i) As demonstrated in the mixing experiment, MSIdetect can identify MSI DNA at much lower concentrations (˜0.1% vs. 3%); and (ii) MSI cancers often have no or minimal large-scale copy-number alterations which are used by iChorCNA to detect the presence of cancer DNA in cfDNA.

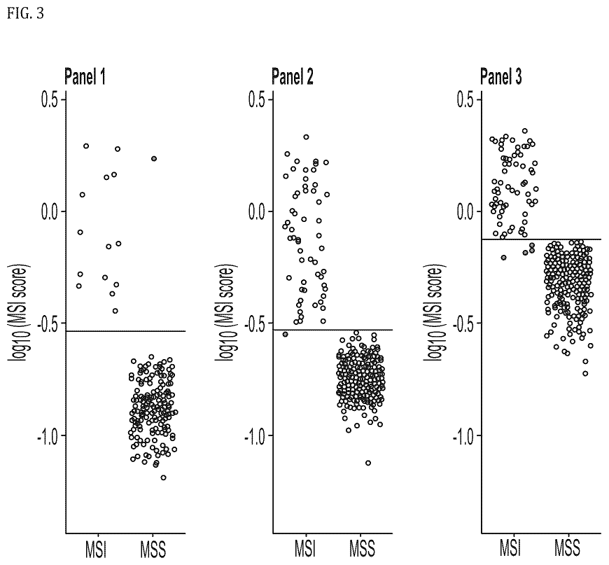

While the development of MSIDetect was geared towards detecting MSI tumors from cfDNA, it can also be applied to DNA sequencing data from tissue samples. As opposed to commonly used clinical MSI classification tools that are based on detecting somatic indels at specific MS loci by comparing the tumor and a matched normal sample, MSIDetect does not require a matched normal since it is based on the patterns of MS-indels throughout the genome. Clinical sequencing panels have the challenge that they usually sequence a small number of genes (tens to a few hundreds) and thus are not covering many MS-loci. However, because MSIDetect can use very shallow sequencing, it can leverage the off target reads generated in standard gene panels. As a proof of concept MSIDetect was applied to a cohort of 1308 panel sequencing data from tissue biopsies, generated using four different versions of the Dana-Farber Cancer Institute OncoPanel assay. The cohort had clinical MSI status derived either based on immunohistochemistry of the four MMR genes or using the Bethesda protocol (5 or 7 markers). Since sequencing noise characteristics and coverage distribution was different across assays, threshold in MSIDetect was tuned for each version of OncoPanel (ie. one parameter per assay). The data was partitioned to a training set (60% of the data) and tested the performance on the remaining 40% of samples. To obtain a robust estimate, the accuracy was averaged across a 100 random partitions to train and test sets, which yielded an average error rate of 0.9% (FIG. 3).

Since MSIDetect can find traces of MSI tumors in cfDNA, even when the tumor DNA represents only ˜0.1% of the cfDNA, it has the potential to serve as an early detection tool or for monitoring relapse of tumors or minimal residual disease (MRD). While some MRD assays can detect cancer in a low cancer DNA fraction (˜10⁻⁵;REF), MSIDetect has the advantage that it does not depend on specific mutations in a patient's tumor. Consequently, it does not depend on sequencing the original tumor or designing patient-specific assays, rather it is based on standard low-pass WGS of the cfDNA.

While MSIDetect was developed as a tool for detecting MSI in cfDNA, it can also serve as a tool for classifying tumors as MSI or MSS, and can be applied to a wide range of input data, including WGS, low coverage WGS, WES and gene panels. The exact threshold separating MSI samples from MSS samples may differ among different sequencing protocols and alignment methods, and thus there is a need to generate controls processed with the same protocol. There is no need to use MSS cases as controls; non-cancerous samples are sufficient. As mentioned above, detection of MSI tumors from cfDNA can be helpful for early detection, which is very important in Lynch syndrome cases. Because ultra low pass sequencing is sufficient for MSIDetect classification, it can be widely implemented in clinics both for MSI classification and MSI detection from the blood. The goal here was to develop a tool that will be simple and inexpensive. MSIDetect performance can be improved by sequencing deeper and by comparing the cfDNA to DNA from normal cells (e.g. peripheral blood mononuclear cells), both of which are likely to enable MSI detection even in lower fractions of cancer DNA.

### Example 2: Comparison of MSIDetect with Other MSI NGS Classifiers

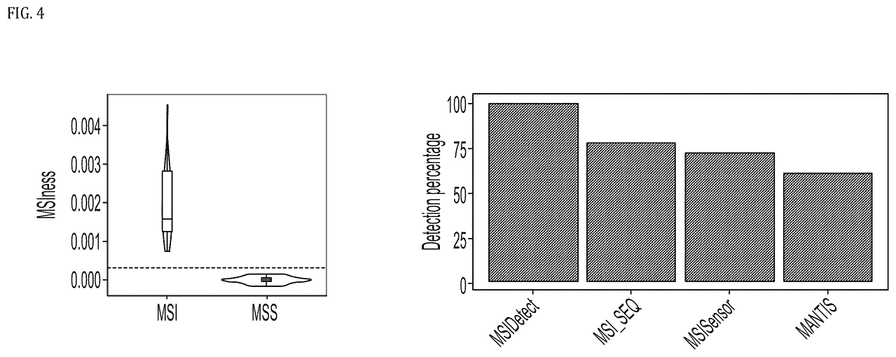

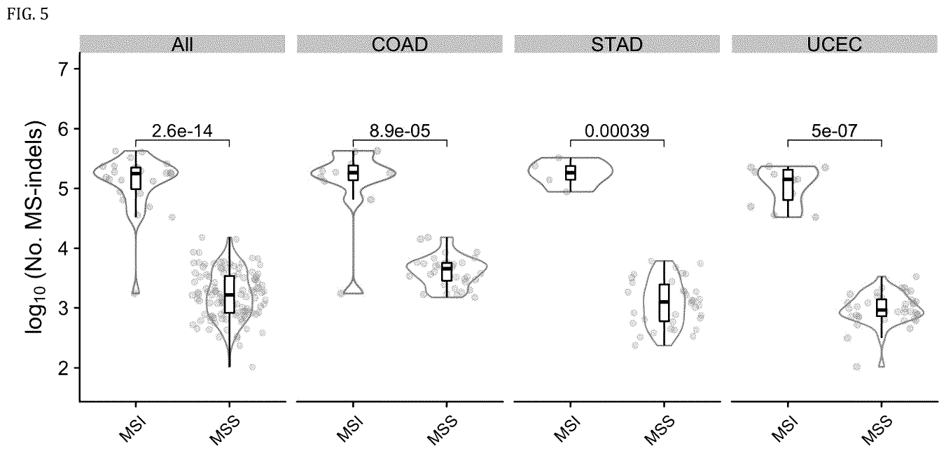

The efficacy of MSIDetect in classifying cancers was compared to the classification methods MSI SEQ, MSISensor, and MANTIS (FIG. 4). Each method was used to detect MSI cancers from a test set comprising both MSI and MSS cancer samples. The MSIness scores of the MSI and MSS cancer samples are presented in the left panel of FIG. 4. As shown in in the right panel of FIG. 4, MSIDetect was more effective than any of the three other programs evaluated in classifying and, therefore, detecting, the MSI cancer samples.

The results described herein were obtained using the following methods and materials.

### Cancer Cell Lines

The Cancer Cell Line Encyclopedia (CCLE) WGS data was analyzed. The MSI status of each cell line was determined based on both the fraction of MS-indels among all indels and the activity levels of MSI-associated SNV mutational signatures. MSMuTect (Ver 2.0)_v2 for each locus the distribution of reads supporting with a given number of repeats was generated.

### Mixing Experiments

Six CCLE cell lines were used, including five MSI (DV-90, HEC-6, HEC-108, SNU-C2A, HEC-59) and one MSS (SK-N-MC). A dilution series (25 ng/ul, 2.5 ng/ul, 0.25 ng/ul and 0.025 ng/ul) was generated for each of the cell lines. Sample concentrations were based on a fluorescent quantitation assay (Quant-iT PicoGreen Assay Kit, Thermo Fisher Scientific). Appropriate volumes of the dilution series samples were spiked into NA12878 (Coriell) genomic DNA at 0.1%, 0.5%, 1%, 2.5%, 5%, 20%, 0% (All NA12878) and 100% (no NA12878) for a total of 200 ng of DNA. These spike-in samples were diluted to 2 ng/ul, in duplicate, and 100 ng (50 ul at 2 ng/ul) of each were mechanically sheared to 150 bp (Covaris, Inc). Libraries were then prepared using the KAPA Hyper Prep Kit (Kapa Biosystems) according to the recommended protocol. Libraries underwent ten amplification cycles. The 96 libraries (8 spike-in fractions, each in duplicate, 6 cell lines) were quantified (PicoGreen Assay Kit), normalized and pooled. The pool was quantified by qPCR (ViiA qPCR system, Thermo Fisher Scientific) and loaded onto a single lane of HiSeqX (standard protocol, Illumina) for ultra-low-pass whole genome sequencing, for an average coverage of per sample with read lengths of 2×151 bp.

### Patient Cell-Free DNA: Massachusetts General Hospital Samples

Peripheral blood draws were collected at Massachusetts General Hospital in accordance with Institutional Review Board-approved protocols, to which patients provided written informed consent. Blood samples were obtained from 22 MSI patients and 22 MSS patients. Whole blood was collected in two 10 mL streck tubes at baseline or progression for cfDNA analysis. Plasma was separated 1-4 days after collection through two different centrifugation steps (1) room temperature for 10 minutes at 1,600×g (2) room temperature for 10 minutes at 3,000×g. Plasma was stored at −80° C. until ctDNA extraction. Tissue was collected from standard of care procedures (biopsies or surgeries) and either formalin-fixed or flash-frozen. Microsatellite instability was confirmed on tissue samples by immunohistochemistry.

### Patient Cell-Free DNA: Weill-Cornell Samples

Peripheral blood draws were collected at Weill-Cornell Precision Oncology clinic in accordance with Institutional Review Board-approved protocols, to which patients provided written informed consent. Twenty five blood and matching tumor samples were obtained from MSI patients, and blood samples were obtained from 15 Lynch syndrome patients from screening colonoscopy clinic. Whole blood was collected in two 10 mL streck tubes for cfDNA analysis. Plasma was separated 1-4 days after collection through two different centrifugation steps (1) room temperature for 10 minutes at 1,600×g (2) room temperature for 10 minutes at 3,000×g. Plasma was stored at −80° C. until ctDNA extraction. Tissue was collected from standard of care procedures (biopsies or surgeries) and either formalin-fixed or flash-frozen. Microsatellite instability was confirmed on tissue samples by MSISensor score >4 from tumor/normal whole exome data.

All the samples were sequenced by the same protocol as the DNA mixing experiments.

### MSMuTect_v2

Msmutect.py is a script that uses pysam and Python's multiprocessing package to leverage the full capacity of the machine it runs on, in order to perform a fast assessment of indel mutations in specific microsatellite loci, given a WGS-aligned BAM file. The script took three mandatory inputs: (i) the aligned BAM file; (ii) a list of the microsatellite loci to inspect and their genomic location in the same genome build used to align the BAM files (using Phobos file format [REF]); and (iii) a name for the output file. The script processed the list of loci in parallel, trying to optimize its runtime, taking into consideration various parameters, including the size of the machine and I/O times. For each locus, the script extracted all aligned reads mapped to that locus. Unaligned, duplicate, supplementary, or low-quality reads were discarded. Reads that did not span at least 10 bp (value can be changed) on each end of the microsatellite were discarded as well. The remaining reads were checked for the number of motif repetitions in their satellite region, and the results were saved into a histogram. Reads that supported either the reference allele or a deletion were added to the histogram immediately. For insertions, it was checked that the insertion was of the same motif as the microsatellite. A flag was raised in cases where some of the indels contained a non-integer number of motif repetitions. The final output file contained the histograms for all supplied loci. Optional inputs could be used to refine the results. -f allowed a change in the default minimum flanking length of each read. -b changed the number of loci processed by each subprocess. -r specified how many bp should be removed at the end of the read in cases where the user desires the search to support shorter reads. -e specified the % of reads that should be randomly discarded in order to support lower total coverage.

### MSIDetect

In order to collect statistics on MS-indels throughout the genome, for each read that overlaps one of the 23,000,000 MS-loci MSMuTect v2 was used to estimate the difference in number of MS repeats with respect to the reference genome. This was begun by running MSMuTect_v2 on an aligned NGS data in the BAM format, in order to get the sample information on each of the 23,000,000 MS-loci. A score was then assigned for that sample based on comparison to the status of the MS-loci to ˜329 cell-lines that have MSI/MSS status. A sample was considered an MSI if its score is above a certain threshold.

Scoring: For each cell-line and for each MS-locus the number of reads supporting a given change in the number of repeats from the reference genome was counted, and thus a length distribution was generated. For each locus all the distributions from the MSI cell-lines were aggregated into one combined distribution and the same for the MSS cell-lines. (FIG. 2A). The histograms were then normalized to one while 10⁻⁵ was added for any number of repeats that had no reads supporting it. For a locus q, the normalized histograms serve as an empirical length distribution, one for MSI L_{q}^{MSI}(j) and one for MSS L_{q}^{MSS}(j).
Then for every read from every MS-locus its log likelihood ratio (LLR) of being MSI vs. MSS

$$log ( \frac{{L}_{q}^{MSI} ( {r}_{i} )}{{L}_{q}^{MSS} ( {r}_{i} )} )$$

was calculated. The LLR of all the reads of each sample was then generated. Because the majority of the reads were not informative to either direction, summation of all the LLR was blurred by the majority of the uninformative reads. Therefore, only the reads that strongly supported the sample being MSI (LLR>15) were used. In addition, in order to improve the LLR analysis only loci with an A motif (i.e., a stretch of consecutive adenines) and only reads that support a deletion were used. The MSI score was defined as:

$$MSI score = log ( \frac{1}{\vert {Ω}_{T} \vert} \cdot {\sum }_{{r}_{i} ∈ Ω} \frac{{L}_{q}^{MSI} ( {r}_{i} )}{{L}_{q}^{MSS} ( {r}_{i} )} ) \qquad ( 2 )$$

Where, rᵢ is the i'th read, Ω is the set of reads included in the analysis, Ω_{T} is the total set of reads and L is the likelihood that a sample is MSI/MSS given the i'th read. This procedure was generated by analyzing the mixing experiment data.

### Analysis of the DFCI Data

In panel sequencing, usually a small genomic territory (100-500 genes) were amplified and sequenced deeply (˜500×coverage). However, the DNA fragments from outside of the amplified territory were still there and would be sequenced. Actually the amount of off-target reads was comparable to the amount of reads obtained from 0.5×WGS. Therefore, the same pipeline developed about to detect MSI tumors in blood can be used to classify samples as MSI vs MSS.

For a set of ˜1,300 had MSI/MSS status was determined based on an analysis of digital records. Cases with an ambiguous classification were excluded. The MSI-score was calculated for every sample in the same way it was calculated for the cfDNA. The DFCI OncoPanel had a few versions, and each had a different number of genes, which affected the baseline MSI-score MSS samples would have. Therefore each version of the panel was analyzed separately. For each version the sample was randomly divided into a training set (60% of the sample) and a test set (40%), of the samples. The value that separated the most between the MSS and MSI cases was found by minimizing the overall false classification rate of the training set. If there was more than one value, the middle of the set of the values was used. The error rate was then evaluated based on the test set. This process was repeated 1000 times to generate the average error rate.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. Citation or identification of any document in this application is not an admission that such document is available as prior art to the present disclosure. In case of conflict, the present specification, including definitions, will control. In addition, the materials, methods, and examples are illustrative only and not intended to be limiting.

What is claimed is:

1. A method for characterizing microsatellite instability in a biological sample, the method comprising comparing the distribution of insertions and deletions in sequencing data obtained from the biological sample over a plurality of microsatellite indels across a genome, wherein the distribution of insertions vs. deletions present in the genome indicates the presence or absence of microsatellite instability in the biological sample.

2. A method for treating a selected subject having a neoplasia characterized as having microsatellite instability, the method comprising administering to the selected subject an immune checkpoint blockade therapeutic, wherein the subject is selected by comparing the distribution of insertions and deletions in sequencing data obtained from a biological sample of the subject over a plurality of microsatellite indels across a genome, wherein the distribution of insertions vs. deletions present in the genome indicates the presence or absence of microsatellite instability in the biological sample.

3. The method of claim 2, wherein the checkpoint blockade therapeutic is a PD-1/PD-L1 inhibitor.

4. The method of claim 1, wherein the biological sample comprises cell free DNA (cfDNA) and/or tissue.

5. The method of claim 1, wherein the biological sample comprises tumor DNA.

6. The method of claim 4, wherein the cell free DNA comprises between about 0.1% and about 3% tumor DNA.

7. The method of claim 1, wherein the sequencing is low coverage whole-genome sequencing, low coverage whole-exome sequencing, or targeted Next-Generation Sequencing.

8. The method of claim 1, wherein the sequence coverage is less than about 1×.

9. The method of claim 1, wherein the sequencing coverage is between about 0.1× and 0.5×.

10. The method of claim 1, wherein the cancer is a colorectal, stomach, or endometrial cancer.

11. The method of claim 1, wherein the cancer is selected from the group consisting of a colon adenocarcinoma, a stomach adenocarcinoma, or a uterine corpus endometrial carcinoma.

12. The method of claim 1, wherein the cancer is a breast, adrenal, or cervical cancer.

13. The method of claim 1, wherein the subject has or is at risk for developing Lynch syndrome.

14. The method of claim 1, wherein the subject has or is at risk for developing minimal residual disease.

15. The method of claim 1, wherein the subject has or is at risk for developing a relapse of a tumor.

16. The method of claim 1, wherein the comparing involves calculating a ratio between insertions and deletions.

17. The method of claim 1, wherein the comparing involves calculating a log likelihood ratio (LLR) of the presence or absence of microsatellite instability in the sample.

18. The method of claim 17, wherein a log likelihood ratio above a threshold value indicates the presence of microsatellite instability in the biological sample.

19. The method of claim 18, wherein the threshold value is at least about log(15).

20. The method of claim 1, wherein the method comprises the simultaneous detection of somatic indels in about 23 million microsatellite loci across an entire genome and/or exome.

21. The method of claim 17, wherein the log likelihood ratio is an MSI score calculated according to the following formula:
$$MSI score = log ( \frac{1}{\vert {Ω}_{T} \vert} \cdot {\sum }_{{r}_{i} ∈ Ω} \frac{{L}_{q}^{MSI} ( {r}_{i} )}{{L}_{q}^{MSS} ( {r}_{i} )} ) , \qquad ( 2 )$$
wherein rᵢ is the i'th read, Ω is the set of reads included in the analysis, Ω_{T} is the total set of reads and L is the likelihood that a sample is MSI/MSS given the i'th read.


SEQUENCE LISTING (5 sequences)

SEQ ID NO: 1 - protein, 288 aa, Homo sapiens
MQIPQAPWPVVWAVLQLGWRPGWFLDSPDRPWNPPTFFPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - DNA, 867 bp, Homo sapiens
  source 1..867: mol_type = other DNA
atgcagatcccacaggcgccctggccagtcgtctgggcggtgctacaactgggctggcggccaggatggttcttagactccccagacaggccctggaacccccccaccttcttcccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagccgacggccctcggagtgcccagccactgaggcctgaggatggacactgctcttggcccctctga

SEQ ID NO: 3 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 4 - DNA, 873 bp, Homo sapiens
  source 1..873: mol_type = other DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 5 - DNA, 10 bp, Homo sapiens
  source 1..10: mol_type = genomic DNA
acacacacac